(19) United States
(12) Patent Application Publication
(10) Pub. No.: US 2010/0092493 A1
(43) Pub. Date: Apr. 15, 2010

(54) Modulation of Complement System Activation for Treatment of Bleeding-Related Inflammation

(75) Inventor: Yang Cao, North Brunswick, NJ (US)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: NOVO NORDISK A/S, Bagsvaerd (DK)

(21) Appl. No.: 12/529,980

(22) PCT Filed: Mar. 6, 2008
(86) PCT No.: PCT/US08/56037

§ 371 Date: Oct. 7, 2009

Related U.S. Application Data
(60) Provisional application No. 60/905,178, filed on Mar. 6, 2007.
(60) Provisional application No. 60/905,356, filed on Mar. 7, 2007.

(51) Int. Cl. A61K 39/395; A61K 38/36; A61K 38/37; A61K 38/48; A61P 29/00

(52) U.S. Cl. 424/175.1; 424/94.5; 424/94.64

(57) ABSTRACT

This invention provides, inter alia, methods and compositions for treating, reducing the incidence of, reducing the severity of, or delaying onset or advancement, of synovitis and/or bleeding-associated inflammation in a subject in need thereof. The compositions comprise, and methods make use of, an effective amount of an agent, which abrogates or diminishes activation of the C5aR complement pathway in said subject, and optionally other therapeutic agents including a coagulation factor, other hemostatic factor, and/or second anti-inflammatory agent.

### REFERENCE TO RELATED PATENT APPLICATIONS

This patent application claims the priority benefit of U.S. Provisional Patent Application Nos. 60/905,178 and 60/905,356, filed Mar. 6, 2007 and Mar. 7, 2007, respectively, the entirety of which are hereby incorporated by reference.

### BACKGROUND OF THE INVENTION

The estimated occurrence for hemophilia A is 1 in 10,000 men, and 1 in 25,000 men for hemophilia B. The major complication of hemophilia is intra-articular bleeding in the joints. More than 90% of patients with severe hemophilia will have joint bleeding unless clotting factor replacement is provided as prophylaxis at young ages. On average, severe hemophilic patients will experience 17 joint bleedings annually. Repeated joint bleeding leads to hemophilic synovitis.

Current treatments include prophylaxis with Factor VIII, however replacement therapy must commence at young ages in order to prevent development of synovitis. When replacement fails, or subjects become resistant to such replacement, on-demand therapies are utilized. Such therapies include administration of pain killers, such as COX-2 inhibitors and ice packs, followed by synovectomy and ultimately total joint replacement. None of the above-described therapies alleviate the inflammatory damage associated with the bleeding, and moreover, concerns with COX-2 inhibitor-mediated cardiovascular risk have resulted in market withdrawal of COX-2 inhibitors. Alternative and/or improved therapeutic approaches for treating bleeding associated inflammation of the joints will likely be of significant benefit to these and other patients.

### SUMMARY OF THE INVENTION

The invention described herein provides several new methods for the treatment of bleeding-associated inflammation, such as synovitis, compositions useful in such treatments, and related methods and compositions. In a first exemplary aspect, this invention provides a method of treating, reducing the incidence of, reducing the severity of, or delaying onset of synovitis, in a mammalian subject in need thereof, the method comprising administering to the subject an effective amount of an agent, which abrogates or diminishes activation of a complement pathway in said subject (i.e., a “complement pathway activation modulating agent”, which may be also referenced as a “CPAMA”, or CPAM agent; if the term “the agent” is used herein, it generally is in reference to a CPAMA unless otherwise stated or clearly contradicted by context). In a related sense, the invention provides for the use of such an agent in the treatment of synovitis or other bleeding-related inflammation and also provides for the use of such an agent in the preparation of a medicament for the treatment of such conditions. In another sense, the invention provides a product (such as a pharmaceutically acceptable composition) comprising a CPAMA for treatment of synovitis (e.g., hemophilic synovitis) or bleeding-related inflammation (which may or may not include synovitis).

In one embodiment, the subject to be treated by the above-described uses/methods suffers from hemophilia (which may be hemophilia A or hemophilia B or both). In another aspect, the subject also or alternatively suffers from another bleeding disorder. In another aspect, the patient may suffer from bleeding-related inflammation, which may or may not include hemophilic synovitis or synovitis (generally).

In one particular embodiment, the CPAM agent is a biologic (or biomolecule—such as protein, glycoprotein, glycan, nucleic acid, lipid, etc., which is produced naturally in an organism or is a variant or derivative of such a molecule), which in one embodiment, is an antibody. In a more specific embodiment, the CPAM agent is an antibody that specifically interacts with an extracellular loop of a C5a receptor (C5aR). In one embodiment, the CPAM antibody also or alternatively specifically inhibits or abrogates C5a binding to its cognate receptor. In a particular exemplary embodiment, the CPAM antibody is produced by a hybridoma having an ECACC accession number 00110609, 02090226, or 02090227, or ATCC accession number HB 11382, HB 11384, or HB-11625. The invention accordingly also provides for the use of such agents in the treatment of such conditions and in the preparation of medicaments for the treatment of such conditions. The invention also provides a product comprising any of these agents for the treatment of synovitis or bleeding-related inflammation (which may or may not include synovitis).

In another embodiment, the agent is an oligopeptide, which typically is limited to a non-antibody peptide, polypeptide, or protein (or a derivative thereof).

In one embodiment, the agent is a small molecule compound. Typically, a small molecule compound (or candidate agent in the context of methods used to identify compounds useful in the practice of the inventive methods described herein) in the context of this invention is a non-peptide small organic compound having a molecular weight of more than about 100 and less than about 2,500 Daltons. Such small molecule compounds/small molecule candidate agents typically comprise functional groups necessary for structural interaction with proteins (e.g., C5a and/or C5aR), such as through hydrogen bonding, and typically include at least an amine, carbonyl, hydroxyl, and/or carboxyl group, and commonly at least two of these functional groups. Such small molecule compounds also typically comprise cyclical carbon or heterocyclic structures and/or aromatic or polyaromatic structures, frequently substituted with one or more of the above-described functional groups. Small molecules also or alternatively may specifically be selected from saccharides, fatty acids, steroids, purines, pyrimidines, and derivatives, structural analogs, or combinations of any thereof. Suitable small molecule compounds typically are organic molecules, preferably small organic compounds having a molecular weight of more than 100 and less than about 2,500 Daltons, comprising at least one amine, carbonyl, hydroxyl and/or carboxyl group, and comprising a cyclical carbon or heterocyclic structure. In a particular exemplary embodiment, the CPAM small molecule agent is selected from N-((4-dimethylaminophenyl)methyl)-N-(4-isopropylphenyl)-7-methoxy-1,2,3,4-tetra-hydronaphthalen-1-carboxamide hydrochloride, R(−)-2-[4′-isobutylphenyl)-propionylamino]-1,1 -dimethylpiperidium iodide, ([R]-2-(4-isobutyl-phenyl)-N-(3-piperidin-1-yl-propyl)-propionamide, and 3-aryl-5,6-disubstituted pyridazine, or combinations of any thereof (which phrase, throughout, also should be understood to include combinations of all thereof). In another aspect, the CPAM small molecule compound is a phenyl amino compound antagonist of C5aR as described in, e.g., WO 2006128670.

In one embodiment, the method further comprises administering a hemostatic agent, such as a coagulation factor to the subject, which in some embodiments is Factor XIII, Factor IX, Factor VII, Factor VIII, fibrinogen, or a biologically active fragment of any thereof, a functional variant of any thereof, or a derivative of any thereof, or combinations of any thereof. In one exemplary embodiment, the method further comprises administering an agent, which promotes or enhances production of a coagulation factor or other hemostatic agent to said subject, which in one embodiment is a nucleic acid encoding said coagulation factor, which in another embodiment encodes Factor XIII, Factor IX, Factor VII, Factor VIII, fibrinogen, thrombin, or a protein antifibrinolytic agent (e.g., aprotinin) or a derivative or variant thereof, or combinations of any thereof.

A CPAM agent can abrogate or diminish activation of any suitable complement pathway(s) in any suitable manner. In one embodiment, the CPAM agent abrogates or diminishes C5a expression, C5a binding to a cognate receptor, C5a receptor signaling, or a combination of any thereof.

In one embodiment, the CPAM agent is administered as part of a pharmaceutical composition, which in one embodiment, is formulated for oral, in joint (local), intravenous, subcutaneous, or topical administration. Thus, in one exemplary embodiment, the invention provides such a pharmaceutical composition, wherein the composition is formulated as an injectable formulation.

The methods of the invention can generally be used to treat any type of synovitis. In one embodiment, the synovitis is acute, or in another embodiment, the synovitis is chronic. In still another embodiment, the synovitis is pre-acute. Compositions comprising such agents can be indicated for use in one or more of these indications and the method provides for the promotion of sales and use of compositions comprising such agents by way of educating patients, health care providers, etc., regarding the use of such compositions for such purposes.

In another aspect, this invention provides a method of treating, reducing the incidence of, reducing the severity of, or delaying onset (or advancement) of bleeding-associated inflammation (which may or may not include synovitis and more particularly may exclude synovitis associated with hemophilia, generally, or a type of hemophilia (e.g., hemophilia A)) in a subject in need thereof, the method comprising administering to the subject an effective amount of agent, which abrogates or diminishes activation of a complement pathway in said subject (i.e., a CPAMA).

In another aspect, the invention provides new pharmaceutical compositions, which may be, e.g., used in the treatment of any of the above-described disorders/conditions. In one exemplary embodiment, this invention provides a pharmaceutical composition comprising an effective amount of an agent, which abrogates or diminishes activation of a complement pathway, such as one of the above-described antibodies (such as one of the anti-C5a antibodies described elsewhere herein), and a coagulation factor or other hemostatic agent (e.g., Desmopressin (DDAVP) or an antifibrinolytic agent such as tranexamic acid and aminocaproic acid).

In another aspect, the invention provides for the use of an agent that inhibits or diminishes activation of a C5a receptor (C5aR) for the treatment of synovitis. The synovitis may be acute, chronic, or sub-acute (and may be associated with one or more types of hemophilia). In a different aspect, the invention provides for the use of such an agent in the preparation of a medicament for treatment of synovitis. In another particular aspect, the agent is an antibody specific for C5aR. In another aspect, the agent is also or alternatively used as a medicament for the treatment of synovitis in patients with hemophilia. In a particular aspect, the agent is an antibody is expressed by or comprises the functional portion of an antibody expressed by a hybridoma having an ECACC accession number 00110609, 02090226 or 02090227, or ATCC accession number HB 11382 or HB 11384, or HB-11625. The invention also provides products (e.g., pharmaceutically acceptable compositions) comprising such agents for the treatment of such conditions (in patients having or at risk of developing such conditions).

In another facet, the invention provides the use of a combination of (a) an agent that abrogates or diminishes activation of C5aR and (b) a coagulation factor or hemostatic factor for the treatment of synovitis. The invention also provides compositions comprising such a combination of elements. In a particular aspect, the coagulation factor or hemostatic factor is selected from the group consisting of Factor XIII, Factor IX, Factor X, Factor XI, Factor VII, Factor VIII, fibrinogen, thrombin, a variant of any thereof, a derivative of any thereof, and combinations of any thereof.

The invention also provides use of an agent that inhibits or diminishes activation of a C5aR for the treatment of synovitis for the treatment of bleeding-associated inflammation. In one aspect, the agent is an antibody specific for C5aR. In another facet, the agent also or alternatively specifically binds an extracellular loop of a C5aR. In an exemplary aspect, the agent is an antibody that is expressed by (or is equivalent to an antibody expressed by) or comprises a functional portion of (e.g., the CDRs of), or consists essentially of, an antibody expressed by a hybridoma having an ECACC accession number 00110609, 02090226 or 02090227, or ATCC accession number HB 11382 or HB 11384, or HB-11625. The invention also provides for the use of such antibodies in the treatment of bleeding-related inflammation and, more particularly (or alternatively) synovitis, such as hemophilic synovitis (and the use of such agents in the preparation of medicaments for such conditions).

In one embodiment, this invention provides a pharmaceutical composition comprising an effective amount of a CPAM agent, which abrogates or diminishes activation of a complement pathway and a nucleic acid encoding a coagulation factor or other hemostatic agent.

In one embodiment, the CPAM agent in the pharmaceutical composition is a biologic (or “biomolecule”), which in one embodiment is an antibody, or in another embodiment is an oligopeptide. In another embodiment, the coagulation factor is Factor VIII, Factor IX, Factor VII, Factor X, Factor XI, an active fragment of any thereof, a variant of any thereof, or a derivative of any thereof.

In another embodiment, the CPAM agent in the pharmaceutical composition abrogates or diminishes C5a expression, C5a binding to a cognate receptor, C5a receptor signaling, or a combination of any or all thereof.

In yet another embodiment, the pharmaceutical composition is formulated for oral, intravenous, subcutaneous, or topical administration, or in another embodiment, the pharmaceutical composition is formulated as an injectable formulation.

These aspects and embodiments are further described in, and additional aspects, embodiments, features, and advantages of the invention (such as, e.g., methods of identifying agents or combinations of agents that are useful in the above-described treatment protocols using test candidates having the characteristics of agents described herein) will be apparent from, the description of the invention provided in this document.

### EXEMPLARY ASPECTS AND FEATURES OF THE INVENTION

To even better illustrate the invention described herein, a nonlimiting list of exemplary aspects and features of the invention is provided here:
1. A method of treating, reducing the incidence of, reducing the severity of, or delaying onset, or delaying advancement of synovitis in a subject in need thereof, the method comprising administering to the subject an effective amount of an agent, which abrogates or diminishes activation of a complement pathway in said subject.
2. The method of aspect 1, wherein said subject suffers from hemophilia.
3. The method of aspect 1 or aspect 2, wherein said agent is an antibody.
4. The method of aspect 3, wherein said antibody specifically interacts with an extracellular loop of a C5a receptor (C5aR).
5. The method of aspect 3 or aspect 4, wherein said antibody specifically inhibits or abrogates C5a binding to its cognate receptor.
6. The method of aspect 4, wherein said antibody is produced by a hybridoma having an ECACC accession number 00110609, 02090226 or 02090227, or ATCC accession number HB 11382 or HB 11384, or HB-11625.
7. The method of any one of aspects 1-6, further comprising administering an effective amount of a coagulation factor or other hemostatic factor to said subject.
8. The method of aspect 7, wherein said factor is Factor XIII, Factor IX, Factor X, Factor XI, Factor VII, Factor VIII, fibrinogen, thrombin, a variant of any thereof, or a derivative of any thereof, or combinations of any thereof.
9. The method of any one of aspects 1-8, wherein said agent abrogates or diminishes C5a expression, C5a binding to a cognate receptor, C5a receptor signaling, or a combination thereof.
10. The method of any one of aspects 1-9, wherein said agent is administered as part of a pharmaceutical composition.
11. The method of aspect 10, wherein said pharmaceutical composition is formulated for intravenous, subcutaneous, or in-joint (local) administration.
12. The method of aspect 10, wherein said pharmaceutical composition is formulated as an injectable formulation.
13. The method of any one of aspects 1-12, wherein said synovitis is acute or sub-acute.
14. A method of treating, reducing the incidence of, reducing the severity of or delaying onset or advancement of bleeding-associated inflammation in a subject in need thereof, the method comprising administering to the subject an effective amount of an agent, which abrogates or diminishes activation of a complement pathway in said subject.
15. The method of aspect 14, wherein said agent is an antibody.
16. The method of aspect 15, wherein said antibody specifically interacts with an extracellular loop of a C5a receptor (C5aR).
17. The method of aspect 15 or aspect 16, wherein said antibody specifically inhibits or abrogates C5a binding to its cognate receptor.
18. The method of aspect 16, wherein said antibody is produced by a hybridoma having an ECACC accession number 00110609, 02090226 or 02090227, or ATCC accession number HB 11382 or HB 11384, or HB-11625.
19. The method of any one of aspects 14-18, further comprising administering an effective amount of a coagulation factor or other hemostatic factor to said subject.
20. The method of aspect 19, wherein said coagulation factor is Factor XIII, Factor IX, Factor VII, Factor X, Factor XI, Factor VIII, fibrinogen, thrombin, or a variant of any thereof, or a derivative of any thereof, or combinations of any thereof.
21. The method of any one of aspects 14-19, wherein the agent abrogates or diminishes C5a expression, C5a binding to a cognate receptor, C5a receptor signaling, or a combination thereof.
22. The method of any one of aspects 14-21, wherein said agent is administered as part of a pharmaceutical composition.
23. A pharmaceutical composition for use in treating bleeding-associated inflammation comprising an effective amount of a combination of (a) an agent, which abrogates or diminishes activation of a complement pathway and (b) a coagulation factor or other hemostatic factor.
24. The pharmaceutical composition of aspect 23, wherein said agent is a monoclonal antibody or an active antibody fragment.
25. The pharmaceutical composition of aspect 24, wherein said antibody specifically interacts with an extracellular loop of a C5a receptor (C5aR).
26. The pharmaceutical composition of aspect 24 or aspect 25, wherein said antibody specifically inhibits or abrogates C5a binding to its cognate receptor.
27. The pharmaceutical composition of aspect 25, wherein said antibody is produced by a hybridoma having an ECACC accession number 00110609, 02090226 or 02090227, or ATCC accession number HB 11382 or HB 11384, or HB-11625.
28. The pharmaceutical composition of any one of aspects 23-27, wherein said coagulation factor is Factor XIII, Factor IX, Factor VII, Factor VIII, fibrinogen, or a variant of any thereof, or a derivative of any thereof, or a combination of any thereof.
29. The pharmaceutical composition of any one of aspects 23-28, wherein said agent abrogates or diminishes C5a expression, C5a binding to a cognate receptor, C5a receptor signaling, or a combination thereof.
30. The pharmaceutical composition of any one of aspects 23-29, wherein said pharmaceutical composition is formulated for subcutaneous, intravenous, or in joint (local) administration
31. The pharmaceutical composition of any one of aspects 23-30, wherein said pharmaceutical composition is formulated as an injectable formulation.

These aspects are more fully described in, and additional aspects, features, and advantages of the invention will be apparent from, the description of the invention provided herein.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a representation of a micrograph of C5aR Expression in Human Normal Synovial Tissue. Immunohistochemistry of human synovium probed with W17/1 (B) versus IgG control (A) is shown.

FIG. 2 is a representation of a micrograph showing the staining of human osteoarthritis synovium by an anti-human C5aR1 antibody in an immunoassay.

FIG. 3 is a representation of a micrograph showing the staining of human hemophilic synovitis tissue by an anti-human C5aR1 antibody in an immunoassay.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The present invention is directed, in some embodiments, to compositions and/or methods for the treatment and/or prevention of damage to diarthrodial (synovial) joints, or bleeding-associated inflammation, or both, in a subject in need thereof (and also the use of such compositions in the treatment of such conditions or in the preparation of medicaments for the treatment of such conditions).

In some embodiments, synovitis is to be understood to encompass any inflammation of the synovial or articular cartilage of the joint, of any etiology. In some embodiments, synovitis or arthropathy may be of autoimmune etiology, inflammation, trauma, or others, any of which represents an embodiment of this invention. In some embodiments, the synovitis is traumatic synovitis, inflammation of the synovial membrane, or damage to the articular cartilage of the joint. In some embodiments, the synovitis is hemophilic synovitis.

In some embodiments, the invention provides compositions for such treatment, which may include a composition specially formulated for intra-articular and/or parenteral use, for use in, e.g., the treatment and/or prevention of synovitis and/or damage to articular cartilage, for example, for post surgical joint lavage or treatment and/or prevention of inflammatory arthritis, osteoarthritis (OA), and/or degenerative joint disease (DJD). Such particular methods of using such compositions are additional aspects of the invention.

In one embodiment, this invention provides a method of treating, reducing the incidence of, reducing the severity of, or delaying onset or advancement of synovitis in a subject in need thereof, the method comprising administering to the subject an effective amount of an agent, which abrogates or diminishes activation of a complement pathway in said subject.

In one aspect, the invention relates to a method of treating one or more diseases, disorders, or conditions, which are associated with bleeding-associated inflammation. In one embodiment, the terms “treating” or “treatment” includes preventative as well as disorder remitative treatment. In some embodiments, the terms “treating” or “treatment” includes “reducing”, “suppressing” and “inhibiting” the disease, disorder, and/or condition, or symptoms associated thereto. In some embodiments, the terms “reducing”, “suppressing” and “inhibiting” have their commonly understood meaning of lessening or decreasing, in another embodiment, or delaying, in another embodiment, or reducing, in another embodiment the incidence, severity or pathogenesis of a disease, disorder, or condition. In embodiment, the term treatment refers to delayed progression of, prolonged remission of, reduced incidence of, and/or amelioration of symptoms associated with the disease, disorder, or condition. In one embodiment, the terms “treating” “reducing”, “suppressing” or “inhibiting” refer to a reduction in morbidity, mortality, or a combination thereof, in association with the indicated disease, disorder, or condition. These terms, and similar concepts in respect of the therapeutic methods of the invention, can be evaluated in terms of an individual or, more commonly, in respect of a population of individuals that have the relevant condition, such as a majority of patients with the condition as determined by a clinical trial. In one embodiment, the term “progression” in respect of a disease or condition refers to an increasing in scope or severity, advancing, growing or becoming worse. In one aspect, the methods of the invention provide a means for reducing or stopping (or reducing the likelihood) of progression of a condition, such as synovitis (e.g., synovitis in a hemophiliac). The term “recurrence” means, in another embodiment, the return of a disease after a remission or a latent state. In one aspect, administering a CPAM agent according to the inventive methods described herein can reduce the likelihood of recurrence of a condition, such as synovitis, in a patient. In one embodiment, the methods of treatment of the invention reduce the severity of the disease. In another embodiment, applying the inventive methods described herein also or alternatively can reduce the symptoms associated with the disease. In yet another embodiment, a method of treatment according to the invention may also or alternatively reduce the number of biomarkers normally expressed during disease in the individual. In general, any method of the invention can optionally include a step of detecting whether the state (e.g., health) of the patient or other subject has been modified (typically improved) by relevant tests known in the art for assessing such matters, such as the severity of a condition, quality of life, etc.

In one embodiment, the term “treating” and its included aspects, refers to the administration to a subject with the indicated disease, disorder, or condition, or in some embodiments, to a subject predisposed to the indicated disease, disorder, or condition (or a subject that has been identified as being in risk, typically imminent risk, of developing the disease, disorder or condition). The term “predisposed to” is to be considered to refer to, inter alia, a genetic profile or familial relationship which is associated with a trend or statistical increase in incidence, severity, etc. of the indicated disease. In some embodiments, the term “predisposed to” is to be considered to refer to inter alia, a lifestyle which is associated with increased risk of the indicated disease. In some embodiments, the term “predisposed to” is to be considered to refer to inter alia, the presence of biomarkers which are associated with the indicated disease. A risk may also exist due to a condition that is not linked with (or known to be linked) with a predisposition, such as a bleeding condition associated with an injury or condition that is or is likely to be associated with inflammation.

In another embodiment, practice of the inventive methods can reduce the pathogenesis of a target disease, condition, or disorder. In some embodiments, the term “reducing the pathogenesis” is to be understood to encompass reducing tissue damage, or organ damage associated with a particular disease, disorder, or condition. In another embodiment, the term “reducing the pathogenesis” is to be understood to encompass reducing the incidence or severity of an associated disease, disorder, or condition, with that in question. In another embodiment, the term “reducing the pathogenesis” is to be understood to encompass reducing the number of associated diseases, disorders, or conditions with the indicated, or symptoms associated thereto.

The term “administering”, or grammatical forms thereof, refers to bringing a subject in contact with a compound of the present invention. Administration can be accomplished in vitro, i.e. in a test tube, or in vivo, i.e., in cells or tissues of living organisms, for example humans (or ex vivo). In one embodiment, the present invention encompasses administering the compounds of the present invention to a mammalian subject, such as a human patient.

In some embodiments, the term “an effective amount of an agent” refers to that amount, which detectably abrogates or diminishes activation of a complement pathway in a subject, such as a human patient (such an amount may generally be referred to as a “physiologically effective amount”). In one aspect, the invention relates to a method of administering a physiological effective amount of a CPAM agent to a subject so as to induce, promote, and/or enhance a physiological effect therein, such as a reduction of bleeding-associated inflammation, inhibition of one or more complement pathways (e.g., inhibition of C5a/C5aR interactions), etc.

In some embodiments, the term “an effective amount” is the amount, which results in the treatment of the indicated disease, disorder, or condition, including any embodiment of “treatment” as described herein. In some embodiments, the term “an effective amount of an agent” refers to that amount, which also or alternatively results in a therapeutic effect, or palliative effect of the compound. Such an amount of an agent (in either or both cases) may be referred to as a “therapeutically effective amount”. An amount of an agent that is effective for prophylaxis or reduction of onset, reduction in likeliness of onset, and the like, can be referred to as a “prophylactically effective amount.” In one embodiment, a therapeutically effective amount of a CPAM agent is provided to a subject so as to promote, induce, or enhance a therapeutic effect (e.g., the treatment of hemophilic synovitis). In another embodiment, a prophylactically effective amount of a CPAM agent is provided to a subject to reduce risk of developing a condition, prolong time to onset of a condition, reduce the likely severity of an imminent condition, etc., in a subject.

The terms “agent” and “compound” are synonymous, and are used interchangeably herein, unless otherwise stated or clearly contradicted by context. The term “compound” refers to a CPAM agent unless otherwise indicated or clearly contradicted by context.

The term “agent” denotes, in some embodiments, a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent” which is a biologically, physiologically, or pharmacologically active substance (or substances) that acts locally or systemically in a subject so as to produce a therapeutically beneficial physiological effect.

The methods of the invention can be applied to any suitable mammal. A “subject” or “host” refers, in some embodiments, to either a human or a non-human mammal. In one embodiment the mammal is a primate. In one embodiment the mammal is a rodent. In one embodiment the mammal is a farm animal and/or household pet. In one embodiment, the mammal is a human patient.

In one aspect, this invention provides methods and/or compositions for the treatment, as described herein, of synovitis, arthropathy, and/or bleeding-associated inflammation in a subject in need thereof, such as a human patient diagnosed as having such a condition.

In one embodiment, the subject suffers from hemophilia. In one embodiment the subject suffers from hemophilia A. In another embodiment, the subject suffers from hemophilia B. In another aspect, the subject suffers from non-hemophilic synovitis (which may be, e.g., associated with occupational or sports injury).

In one embodiment, the CPAM agent abrogates or diminishes activation of the complement pathway, which in one embodiment is the classical pathway, or in another embodiment is the alternative pathway, or in another embodiment, the agent abrogates or diminishes activation of both pathways.

In one embodiment, the agent abrogates or diminishes (a) C1 activation; (b) cleavage of portions of both of components C4 and C2; (c) formation of the activated complex C4b2a; (d) cleavage of C3; (e) production of C3b; (f) formation of C4b2a3b; (g) cleavage of C5; (h) formation of the membrane attack unit; or (i) a combination of any thereof.

In one embodiment, the agent abrogates or diminishes C3b production, e.g., by abrogating or diminishing (a) cleavage of C3 into C3a and C3b; (b) formation of C3bBb, cleavage of C5 into C5a and C5b and/or initiation of the membrane attack unit; (c) combination of C5b with C6 and C7 to form C5bC6C7; (d) combination of C5bC6C7 with C8; (e) combination with a plurality of C9's to form a further, terminal membrane attack complex, which on the surface of a cell causes cytolysis; or (f) combinations of any thereof. In one aspect, the agent abrogates or diminishes C3 (either by modulation of the functioning of C3 and/or the C3 receptor), C1, and/or C5.

A CPAM agent can generally be any suitable agent that reduces complement activation in a manner that reduces bleeding-associated inflammation. Suitability can be determined by an ordinarily skilled scientist/practitioner in terms of toxicity, effectiveness in promoting the desired physiological or therapeutic effect, and the like. Such agents may comprise antibodies, peptides, inhibitors, antagonists, or others, known in the art, for example as described in Makrides, S. C. Pharmacological Reviews 50:59-78 (1998); U.S. Pat. No. 5,135,916; 3,3′-ureylenebis[6-(2-amino-8-hydroxy-6-sulfp-1-naphthylazo) benzenesulfonic acid, tetrasodium salt, Suramin sodium, tranexamic acid, heparin, hydroxamic acid derivative (e.g., salicylhydroxamic acid (SHA) or acetohydroxamic acid (AHA)); a substituted amide; a substituted hydroxylamine; an aldoxime (e.g., salicylaldoxime); a hydroxyphenyl compound (e.g., catechin or hydrocaffic acid); a terpene; oleanolic acid; and an ephedra. Complement inhibitors are currently available commercially from, for example, from Parke-Davis, Ann Arbor, Mich., USA, and Behringer AG, Morburg, Germany; an aptamer as described in United States Patent Application Publication Number 20060105980, and others, known to those skilled in the art.

In another embodiment, the CPAM agent is a biologic, which in one embodiment, is an agent, which specifically abrogates or diminishes C5a expression, C5a binding to a cognate receptor, C5a receptor signaling, or a combination thereof. In some embodiments, such an agent is an antibody, which in some embodiments specifically interacts with an extracellular loop of a C5a receptor (C5aR), or in some embodiments, specifically inhibits or abrogates C5a binding to its cognate receptor. In a particular embodiment, the CPAM agent is an antibody (which may be an antibody fragment, antibody derivative, etc.) or other molecule (e.g., an antibody mimetic) that specifically binds to an extracellular loop of C5aR that is not located at or near the N-terminus of C5aR. In an even more particular embodiment, the CPAM agent is an antibody or other molecule that binds to the second loop of C5aR. Examples of such antibodies are described in, e.g., WO 03/062278 and US 200524406. Other molecules that may be useful CPAM agents and related or otherwise relevant principles, methods, etc., are described in, e.g., Valentino et al., infra, Lee et al., infra, Schnathaum et al., Bioorganic & Medicinal Chemistry Letters 16 (2006) 5088-5092, Scola et al., JBC Papers in Press (Manuscript M609178200—published on-line on Dec. 11, 2006, at http://www.jbc.org/cgi/doi/10.1074/jbc.M609178200); and Rinder et al., Ann. Thorac. Surg. 2007; 83:146-52. Relevant principles to the practice of the inventive methods are also described in, e.g., Dunn et al., Curr. Opin. Hematol. 12:390-394 (2005); Roosendaal et al., Seminars in Thrombosis and Hemostasis, 29 (1):37-42; Arumugam et al., Clinica Chmimica Acta 374 (2006) 33-45; and Tsoukas et al., Blood, 107 (5):1786-1790 (2006).

In a particular exemplary embodiment, the antibody is produced by a hybridoma having an ECACC accession number 00110609, 02090226 or 02090227, or ATCC accession number HB 11382 or HB 11384, or HB-11625.

In some embodiments, the antibody is as described in United States Patent Application Publication Number 20030175267, or in another embodiment, the antibody is as described in United States Patent Application Publication Number 20050244406, or in another embodiment, the antibody is as described in U.S. Pat. No. 5,480,974, or in another embodiment, the antibody is as described in U.S. Pat. No. 5,177,190, or as described in Japanese Patent Number JP 08109200 A, or as described in World Intellectual Property Organization Publication Number WO 9516033, or as described in Thomas, T C et al., Mol Immunol 1996; 33: 1389-1401. In some embodiments, the methods/compositions of this invention make use of combinations of such antibodies, or combinations of such antibodies and other agents as described herein, any combination which is to be considered as part of this invention.

Antibodies against complement receptors or ligands therefore can be produced and identified using standard methods well known in the art, only some of which are exemplified here. In some embodiments, the antibodies may be prepared by routine means for preparing and characterizing antibodies known in the art (See, e.g., ANTIBODIES: A LABORATORY MANUAL, Cold Spring Harbor Laboratory, 1988).

In some embodiments, the methods for generating monoclonal antibodies (MAbs) begin by immunizing an animal with an immunogenic composition in accordance with the present invention, either with or without prior immunotolerizing, depending on the antigen composition and protocol being employed (e.g., tolerizing to a normal cell population and then immunizing with a tumor cell population), and collecting antisera from that immunized animal. In some embodiments, the antibody is a polyclonal antibody.

A wide range of animal species can be used for the production of antisera. Typically the animal used for production of anti-antisera is a rabbit, a mouse, a rat, a hamster, a guinea pig or a goat. Because of the relatively large blood volume of rabbits, a rabbit is often used, though not exclusively used, for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin, or rabbit serum albumin, can also be used as carriers. Means for conjugating a polypeptide to a carrier protein are well known in the art and include glutaraldehyde, m-maleimidobencoyl-N-hydroxysuccinimide ester, carbodiimyde, and bis-biazotized benzidine.

As is also well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants, and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, may also be given. The process of boosting and titering is repeated until a suitable titer is achieved. When a desired titer level is obtained, the immunized animal can be bled and the serum isolated and stored, and/or the animal can be used to generate MAbs.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified tumor cell or vascular endothelial cell protein, polypeptide, peptide, or intact cell composition. The immunizing composition is administered in a manner effective to stimulate antibody producing cells. Rodents such as mice and rats are preferred animals, however, the use of rabbit, sheep frog cells is also possible. The use of rats may provide certain advantages, but mice are preferred, with the BALB/c mouse being most preferred as this is most routinely used and generally gives a higher percentage of stable fusions.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art. For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210. RCY3, Y3-Ag 1.2.3, IR983F, 4B210 or one of the above listed mouse cell lines; and U-266, GM1500-GRG2, LICR-LON-HMy2 and UC729-6, are all useful in connection with human cell fusions.

Methods for generating hybrids of antibody-producing spleen or lymph node cells and myeloma cells usually comprise mixing somatic cells with myeloma cells in a 4:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (Nature, 256:495-497, 1975; Eur. J. Immunol., 6:511-519, 1976), and those using polyethylene glycol (PEG), such as 37% (v/v) PEG, by Gefter et al. (Somatic Cell Genet., 3:231-236, 1977). The use of electrically induced fusion methods is also appropriate (In: Monoclonal Antibodies: Principles and Practice, 2d Ed., Academic Press, Orlando, Fla., pp. 71-74, 1986).

Fusion procedures may produce viable hybrids at low frequencies, about 1×10⁻⁶ to 1×10⁻⁸. However, the viable, fused hybrids may be differentiated from the parental, unfused cells by culturing in a selective medium. The selective medium may contain an agent that blocks the de novo synthesis of nucleotides in the tissue culture media. In some embodiments, such agents comprise aminopterin, methotrexate, azaserine, or others.

In some embodiments, the selection medium is HAT.

In some embodiments, the culturing provides a population of hybridomas from which specific hybridomas are selected. In some embodiments, selection of hybridomas is performed by culturing the cells by single-clone dilution in microtiter plates, followed by testing the individual clonal supernatants (after about two to three weeks) for the desired reactivity. In some embodiments, the assay may comprise radioimmunoassay, enzyme immunoassay, cytotoxicity assay, plaque assay, dot immunobinding assay, and the like.

In some embodiments, the selected hybridomas are serially diluted and cloned into individual antibody-producing cell lines, which clones can then be propagated indefinitely to provide MAbs. In some embodiments, cell lines may be exploited for MAb production as follows: a sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide MAbs in high concentration. In some embodiments, the individual cell lines are cultured in vitro, where the MAbs are naturally secreted into the culture medium from which they can be readily obtained in high concentration. In some embodiments, MAbs produced by either means are further purified, if desired, using filtration, centrifugation and various chromatographic methods such as HPLC or affinity chromatography.

In some embodiments, the use of a molecular cloning approach to generate monoclonals may be accomplished. For this, combinatorial immunoglobulin phagemid libraries are prepared from RNA isolated from the spleen of the immunized animal, and phagemids expressing appropriate antibodies are selected by panning using cells expressing the antigen and control cells e.g., normal-versus-tumor cells.

In some embodiments, the MAbs employed in the present invention, are of human, murine, monkey, rat, hamster, chicken or even rabbit origin. The invention contemplates the use of human antibodies, “humanized” or chimeric antibodies from mouse, rat, or other species, bearing human constant and/or variable region domains, and other recombinant antibodies and fragments thereof.

In some embodiments, the MAbs for use in compositions and/or methods of this invention refers to the use of functional fragments of a monoclonal antibody. In general, any suitable type of antibody fragment can be used in practice of the inventive methods described herein. In some embodiments, Fab fragments are used. In some embodiments, Fab fragments are obtained by proteolysis of the whole immunoglobulin by the non-specific thiol protease, papain. In some embodiments, Papain is activated by reducing the sulphydryl group in the active site with cysteine, 2-mercaptoethanol or dithiothreitol. Heavy metals in the stock enzyme may be removed by chelation with EDTA (2 mM) to ensure maximum enzyme activity. Enzyme and substrate may be mixed together in the ratio of 1:100 by weight. After incubation, the reaction can be stopped by irreversible alkylation of the thiol group with iodoacetamide or simply by dialysis. The completeness of the digestion may be monitored by SDS-PAGE and the various fractions separated by protein A-Sepharose or ion exchange chromatography.

In some embodiments, the functional fragments are F(ab′)₂ fragments. In some embodiments, the F(ab′)₂ fragments are prepared from IgG of rabbit and human origin via limited proteolysis by the enzyme pepsin. The conditions may be varied as a function of the immunoglobulin subclass.

In some embodiments, the functional fragment is an Fc fragment, or a single chain Fv fragment, whose preparation is well known in the art.

In one embodiment, the agent, which inhibits a complement pathway, is a peptide of any length, or a protein. A peptide or polypeptide typically refers to a single amino acid polymer chain-based compound, whereas a protein may comprise multiple associated polypeptide chains and/or multiple protein units (e.g., a protein may be a dimer or higher order multimer). In some embodiments, the agent is an oligopeptide. In some embodiments, the term “oligopeptide” refers to a peptide of any length, which comprises a functional material, such as a protein, or functional fragment thereof.

In another embodiment, peptides may comprise fragments of native polypeptides from any animal species (including humans), derivatives of native (human and non-human) polypeptides and their fragments, and variants of native polypeptides. In one embodiment, “fragments” comprise regions within the sequence of a mature native polypeptide, and in another embodiment, “derivatives” comprise amino acid sequence and glycosylation variants, and covalent modifications of a native polypeptide. In another embodiment, “variants” refers to amino acid sequence and glycosylation variants of the native polypeptide.

In some embodiments, the CPAM agent is a cyclic peptide C5aR antagonist, such as AcF-[OP(D-Cha)WR], H67KDMQLGR74, YSFKPMPLaR, NMeFKP, NMePHe-Lys-Pro-D-Cha-X-D-Arg, NMe-FKP, NMeFKPdChaWr, Hydrocinnamate-[OP(D-Cha)WR], (F-[OP(D-Cha)WR]), CHIPS peptides or analogues thereof, jun/fos-A8 (jun/fos-C5a-(1-68)-Arg69Ser70Leu71Leu72 Arg73, A8Delta71-73 (C5a-(1-68)-Arg69Ser70), or a combination of any thereof. In some embodiments, the agent is corpstatin, or other C3 inhibitors. These agents are known in the art and described specifically in, e.g., WO 03033528A1, US 20060160726A1, US 20060217530A1, and WO 04040000.

In some embodiments, the CPAM agent is N-((4-dimethylaminophenyl)methyl)-N-(4-isopropylphenyl)-7-methoxy-1,2,3,4-tetra-hydronaphthalen-1-carboxamide hydrochloride, R(−)-2-[4′-isobutylphenyl)-propionylamino]-1,1-dimethylpiperidium iodide, ([R]-2-(4-isobutyl-phenyl)-N-(3-piperidin-1-yl-propyl)-propionamide, 3-aryl-5,6-disubstituted pyridazine, 6-amidino-2-naphthyl 4-guanidobenzoate, or any combination thereof, or in some embodiments, a combination of such agents with any other agent as herein described.

In some embodiments, the CPAM agent is as described in U.S. Pat. No. 6,355,245, or in another embodiment, the agent is as described in U.S. Pat. No. 5,853,722, or in another embodiment, the agent is as described in U.S. Pat. No. 6,074,642, or in another embodiment, the agent is as described in U.S. Pat. No. 6,723,743, or in another embodiment, the agent is as described in United States Patent Application Publication No. 2005/0004031, or in another embodiment, the agent is as described in United States Patent Application Publication No. 2003/0175267, or in another embodiment, the agent is as described in United States Patent Application Publication No. 2005/0226870, or in another embodiment, the agent is as described in World Intellectual Property Organization Publication Number WO 2005/092366, or in another embodiment, the agent is as described in World Intellectual Property Organization Publication Number WO 2006/004589, or in another embodiment, the agent is as described in World Intellectual Property Organization Publication Number WO 2003/082826, or in another embodiment, the agent is as described in World Intellectual Property Organization Publication Number WO 2004/043925, or in another embodiment, the agent is as described in World Intellectual Property Organization Publication Number WO 2003/061765, or in another embodiment, the agent is as described in World Intellectual Property Organization Publication Number WO 2003/009803, or in another embodiment, the agent is as described in World Intellectual Property Organization Publication Number 2005/074607, or in another embodiment, the agent is as described in World Intellectual Property Organization Publication Number WO 2004/022096, or in another embodiment, the agent is as described in World Intellectual Property Organization Publication Number WO 2003/084524, or in another embodiment, the agent is as described in World Intellectual Property Organization Publication Number WO 2003/082829, or in another embodiment, the agent is as described in World Intellectual Property Organization Publication Number WO 2003/082828, or in another embodiment, the agent is as described in World Intellectual Property Organization Publication Number WO 2002/49993, or in another embodiment, the agent is as described in World Intellectual Property Organization Publication Number WO 2004/018460, or in another embodiment, the agent is as described in World Intellectual Property Organization Publication Number WO 2005/007087, or as described in Makrides S C, Pharmacol Rev 1998; 50: 59-87, or as described in Morikis, D. et al., Protein Sci 1998; 7: 619-627; or as described in Pellas T C, et al. J. Immunol 1998; 160: 5616-5621; or as described in Riley R D et al., J Thorac Cardiopvasc Surg 2000; 120: 350-358, each of which is fully incorporated herein by reference. In another embodiment, the agent is a combination thereof, or combinations of these and any other agent or agents as herein described.

In some embodiments, the CPAM agent inhibits expression of genes associated with the complement pathway. In some embodiments, the agent is a nucleic acid

In one embodiment, the term “nucleic acid” molecule refers to DNA or RNA which may be single or double stranded. The nucleic acid may comprise a sequence that is prokaryotic or eukaryotic mRNA in nature. The DNA may comprise cDNA from eukaryotic or prokaryotic mRNA, genomic DNA sequences from eukaryotic or prokaryotic DNA, or, in another embodiment, synthetic DNA sequences. The term also refers to sequences that include any of the known base analogs of DNA and RNA. The nucleic acid sequences within the compositions of this invention may be naked nucleic acid, or, in another embodiment, may be contained within a vector.

According to this aspect of the invention, and in one embodiment, polynucleotide segments encoding sequences of interest can be ligated into commercially available expression vector systems suitable for transducing/transforming mammalian cells and for directing the expression of recombinant products within the transduced cells. It will be appreciated that such commercially available vector systems can easily be modified via commonly used recombinant techniques in order to replace, duplicate or mutate existing promoter or enhancer sequences and/or introduce any additional polynucleotide sequences such as for example, sequences encoding additional selection markers or sequences encoding reporter polypeptides (for further detail see, for example, “Methods in Enzymology” Vol. 1-317, Academic Press, Current Protocols in Molecular Biology, Ausubel F. M. et al. (eds.) Greene Publishing Associates, (1989) and in Molecular Cloning: A Laboratory Manual, 2nd Edition, Sambrook et al. Cold Spring Harbor Laboratory Press, (1989), or other standard laboratory manuals).

In one embodiment, the nucleic acids and/or vectors of this invention comprise, inter alia, a ribozyme, an antisense oligonucleotide or an miRNA agent specific for a coding sequence.

The term “miRNA agent” refers, in one embodiment, to an agent that modulates expression of a target gene by an RNA interference mechanism.

An miRNA agent used in the methods of this invention generally can have any suitable structure. In one embodiment the miRNA agent comprises double-stranded RNA, which can form a hairpin structure. The miRNA agents employed, in another embodiment, are small ribonucleic acid molecules, or oligoribonucleotides, that are present in duplex structures, such as, in one embodiment, two distinct oligoribonucleotides hybridized to each other, or in another embodiment, a single ribooligonucleotide that assumes a hairpin structure to produce a duplex structure.

In one embodiment, the nucleic acids/oligonucleotides comprising may be synthesized on an Applied Bio Systems oligonucleotide synthesizer according to specifications provided by the manufacturer. In another embodiment, the nucleic acids/oligonucleotides or modified oligonucleotides may be synthesized by any number of means as is generally known in the art.

In one embodiment, the term “coding sequence” refers to a nucleic acid sequence that “encodes” a particular polypeptide or peptide. In one embodiment, the coding sequence is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

In one embodiment the term “coding sequence”, includes DNA sequences that encode a polypeptide, as well as DNA sequences that are transcribed into inhibitory antisense molecules.

In one embodiment, the term “reducing expression”, as it refers to vectors, nucleic acids and their use in compositions and/or according to the methods of this invention, refers to a diminishment in the level of expression of a gene when compared to the level in the absence of the described agent.

In one embodiment, reduced expression may be affected at the transcriptional or translational level, or a combination thereof.

According to this aspect of the invention, reduced expression using the compositions and/or according to the methods of this invention, is specific. In one embodiment, the reduction in expression is via an ability to inhibit a target gene without manifest effects on other genes of the cell. The consequences of inhibition can be confirmed, in other embodiments, by examination of the outward properties of the cell or organism or by biochemical techniques such as RNA solution hybridization, nuclease protection, Northern hybridization, gene expression monitoring with a microarray, antibody binding, enzyme linked immunosorbent assay (ELISA), Western blotting, radioimmunoassay (RIA), other immunoassays, and fluorescence activated cell analysis (FACS).

Uptake of the nucleic acid within a cell can be assessed by standard approaches routinely used in the art. For example, filter hybridization techniques (e.g., Northern or Southern blotting) may be performed on cells or tissue contacted with, or from samples isolated from individuals administered the compositions of this invention, or, in another embodiment, RNase protection or reverse transcriptase-polymerase chain reaction (RT-PCR) may be performed, to detect uptake of the plasmids or DNA, and measure expression thereof. The gene product can be detected by an appropriate assay, for example by immunological detection of a produced protein, such as with a specific antibody, or by a functional assay to detect a functional activity of the gene product, such as an enzymatic assay. If the gene product of interest to be expressed by a cell is not readily assayable, an expression system can first be optimized using a reporter gene linked to the regulatory elements and vector to be used. The reporter gene encodes a gene product, which is easily detectable and, thus, can be used to evaluate efficacy of the system. Standard reporter genes used in the art include genes encoding β-galactosidase, chloramphenicol acetyl transferase, luciferase and human growth hormone, or any of the marker proteins listed herein.

In some embodiments, the genes, whose expression is diminished or abrogated comprise sequences encoding a complement component, such as those listed in NCBI's GenBank database, Accession Numbers AAA72968, AAH22312, NP_{—}001726, CAH70782, NP_{—}000195, NP_{—}001728, AAH43484, 1006226A, CAA60121, AAH63851, BAD02321, AAA85332, AAB59537, Q07021, CAI17449, CAM24859, CAI41720, CAM25395, CAA69849, AAA59651, CAA50994, AAA51925, AAA51856, AAA51855, AAH63289, CAI41856, AAM10002, AAD15289, AAA99717, AAA51889, CAB66087, ABD48959, AAR89906, AAA51890, AAA35617, CAA72407, AAA35722, BAB63292, P01024, P0C0L4, P0C0L5, P01031, P00736, P00751, P06681, P09871, P02748, P07358, P07357, or comprises a sequence as set forth in DQ895516, DQ894720, DQ894514, DQ894443, DQ894054, DQ891410, DQ891257, NM_{—}000064, or a sequence encoding a complement receptor, such as those listed in NCBI's GenBank database, Accession Number DQ894940, DQ895197, CAI16725, CAI16724, CAI16723, CAI16044, CAI16043, CAI16042, AAB60694, A34924, CAA48779, AAA99005, AAA99004, AAA37478, AAA37477, AAA37447, AAD15289, AAB60695, AAB60694, NP_{—}004045, NP_{—}004045, NP_{—}001002029, NP_{—}001727, AAD14919, CAA40530, CAA68674, or others as known in the art, or sequences homologous thereto, or suppression or abrogation of combinations thereof.

In one embodiment, the compositions/methods of this invention further comprise/make use of a coagulation factor or other agent/factor that reduces bleeding and/or that promotes hemostasis (a “hemostatic factor” or “hemostatic agent”), which is administered to the subject in need thereof. In some embodiments, the coagulation or hemostatic factor is Factor XIII, Factor IX, Factor VII, Factor VIII, Factor X, fibrinogen, thrombin, antiplasmin (α2 anti-plasmin, TAFI) or an anti-fibrinolytic (e.g., aprotinin) or a derivative or variant thereof, or combinations thereof. In some embodiments, such Factors may comprise a naturally occurring or recombinant form, or a derivative or variant thereof.

In some embodiments, a composition or method of the invention comprises a Factor VIII protein or polypeptide in addition to a CPAM agent. A Factor VIII protein (or “Factor VIII” or “FVIII”) may comprise a naturally occurring or recombinant form of Factor VIII, such as, for example, that disclosed in U.S. Pat. No. 5,563,045, U.S. Pat. No. 5,451,521, U.S. Pat. No. 5,422,260, U.S. Pat. No. 5,004,803, U.S. Pat. No. 4,757,006, U.S. Pat. No. 5,661,008, U.S. Pat. No. 5,789,203, U.S. Pat. No. 5,681,746, U.S. Pat. No. 5,595,886, U.S. Pat. No. 5,045,455, U.S. Pat. No. 5,668,108, U.S. Pat. No. 5,633,150, U.S. Pat. No. 5,693,499, U.S. Pat. No. 5,587,310, U.S. Pat. No. 5,171,844, U.S. Pat. No. 5,149,637, U.S. Pat. No. 5,112,950, U.S. Pat. No. 4,886,876, WO 94/11503, WO 87/07144, WO 92/16557, WO 91/09122, WO 97/03195, WO 96/21035, WO 91/07490, EP 0 672 138, EP 0 270 618, EP 0 182 448, EP 0 162 067, EP 0 786 474, EP 0 533 862, EP 0 506 757, EP 0 874 057, EP 0 795 021, EP 0 670 332, EP 0 500 734, EP 0 232 112, EP 0 160 457, Sanberg et al., XXth Int. Congress of the World Fed. Of Hemophilia (1992), and Lind et al., Eur. J. Biochem., 232:19 (1995), all of which are incorporated by reference in their entirety. In one aspect, the coagulation factor combined with a CPAM agent in a method or composition of the invention is a “factor VIII polypeptide.” The term “factor VIII polypeptide” encompasses, without limitation, factor VII, as well as factor VIII-related polypeptides. The term “Factor VIII” is generally intended to encompass, without limitation, polypeptides having the amino acid sequence as described in Toole et al.; Nature 1984; 312: 342-347 (wild-type human factor VIII) and allelic variants thereof. “Factor VIII-related polypeptides” (which may be used as a substitute for FVIII) can include, without limitation, factor VIII polypeptides that have either been chemically modified relative to human factor VIII and/or contain one or more amino acid sequence alterations relative to human factor VIII (i.e., factor VIII variants), and/or contain truncated amino acid sequences relative to human factor VIII (i.e., factor VIII fragments). Typically, variants of Factor VIII, like variants of other coagulation factors, to be used in the methods/compositions described herein, will have high levels of identity in terms of amino acid sequence composition to the related/corresponding human protein (e.g., at least about 80%, 85%, 90%, 92%, 93%, 95%, 97%, or 99% identity thereto—in this case to human Factor VIII) and retain at least substantially similar activity to the human protein (e.g., in terms of regulating bleeding or as tested in a relevant assay (e.g., a Factor VIII activity assay is described in US 20030199444)). The term “factor VIII-related polypeptides” are intended to encompass such polypeptides in their uncleaved (zymogen) form, as well as those that have been proteolytically processed to yield their respective bioactive forms, which may be designated “factor VIIIa-related polypeptides” or “activated factor VIII-related polypeptides”. The phrase “factor VIII-related polypeptides” encompasses, without limitation, polypeptides exhibiting at least a significant proportion of, substantially the same or improved biological activity relative to wild-type human factor VIII. Non-limiting examples of factor VIII polypeptides include plasma-derived human factor VIII as described, e.g., in Fulcher et al.; Proc. Acad. Nat. Sci. USA 1982; 79:1648-1652, and Rotblat et al.; Biochemistry 1985; 24:4294-4300, and plasma-derived porcine FVIII as described, e.g., in Fass et al.; Blood 1982; 59: 594-600 and Knutson et al.; Blood 1982; 59: 615-624. Non-limiting examples of factor VII sequence variants are described, e.g., in Lollar et al.; Blood 2000; 95(2): 564-568 (hybrid porcine/human FVIII polypeptides) and Lollar et al.; Blood 2001; 97 (1): 169-174. Commercially available FVIII products (so-called replacement products) are typically derived from normal pooled plasma or genetically engineered mammalian cell lines. Replacement products are often classified according to final purity, defined as specific activity (international units of clotting factor activity per mg of protein, IU/mg). Intermediate products have relatively low specific activity (<50 IU/mg) because they also contain extraneous plasma proteins, such as fibrinogen, fibronectin and other non-coagulant proteins. High purity (>50 IU/mg) and ultra high purity (>3000 IU/mg) contain little or virtual no other plasma proteins other that albumin added as a stabilizer. Non-limiting examples of commercially available factor VIII products (concentrates and preparations) that may be used according to the present invention are, for example, without limitation, ReFacto (B-domain deleted rFVIII) from AHP/Genetics Institute, Alphanate (FVIII) from Alpha, Bioclate (rFVIII) from Aventis, Monoclate-P (factor VIII:C) from Aventis, Helixate (rFVIII) from Aventis, Recombinate (rFVIII) from Baxter, Hemofil M (FVIII) from Baxter, Kogenate (rFVIII) from Bayer, Nordiate (FVIII) from HemaSure, FACTEUR VIII-LFB (human plasma-based FVIII) from Laboratoire Francais du Fractionnement et des Biotechnologies (LFB), Hyate:C (porcine FVIII) from Speywood, and Kogenate SF (sucrose formulated rFVIII) from Bayer. Nonlimiting examples of high and ultra high activity products are Alphanate™ (Alpha) (low); ReFacto™ (AHP/Genetics Institute), Kogenate™ SF (Bayer), Kogenate™ (Bayer), Helixate™ (Aventis), Recombinate™ (Baxter), Monoclate-P™ (Aventis), Hemofil™ M (Baxter) (all ultra high).

As indicated above, in another embodiment a composition or method of the invention can include a combination of a Factor IX polypeptide and a CPAM agent. A Factor IX protein can be human recombinant Factor IX (rhFIX). A Factor IX protein can more particularly be selected from BeneFix™ (AHP/Genetics Institute), Mononine™ (Aventis), Proplex™ (Baxter), Bebulin VH (Baxter), FACTEUR IX-LFB (Laboratoire Francais du Fractionnement et des Biotechnologies (LFB)), Immunine (Baxter/Immuno), Octanyne (Octapharma), Octanine F (Octapharma), Mono FIX-VF (CSL), or Novact M (Kaketsuken). In general, the phrase “factor IX polypeptide” encompasses, without limitation, Factor IX, as well as Factor IX-related polypeptides (either of which may generally be used/incorporated in the methods or compositions of the invention). The term “factor IX” is intended to encompass, without limitation, polypeptides having the amino acid sequence as described in Jaye et al., Nucleic Acids Res. 1983 (wild-type human factor IX) and natural allelic variations of human factor IX that may exist and occur from one individual to another. Also, degree and location of glycosylation or other post-translation modifications may vary depending on the chosen host cells and the nature of the host cellular environment. The term “Factor IX” is also intended to encompass Factor IX polypeptides in their uncleaved (zymogen) form, as well as those that have been proteolytically processed to yield their respective bioactive forms, which may be designated Factor IXa (as is the case with other coagulation factors described herein). “Factor IX-related polypeptides,” which can be incorporated in the methods and compositions of the invention, include, without limitation, factor IX polypeptides that have either been chemically modified relative to human factor IX and/or contain one or more amino acid sequence alterations relative to human factor IX (i.e., factor IX variants), and/or contain truncated amino acid sequences relative to human factor IX (i.e., factor IX fragments). Typically, variants of Factor IX, like variants of other coagulation factors, to be used in the methods/compositions described herein, will have high levels of identity to the related human protein (e.g., at least about 80%, 85%, 90%, 92%, 93%, 95%, 97%, or 99% identity thereto) and retain at least substantially similar activity to the human protein (e.g., in terms of regulating bleeding or as tested in a relevant assay (e.g., a Factor IX assay is described in US 20030203845)). Non-limiting examples of factor IX polypeptides include plasma-derived human factor IX as described, e.g., in Chandra et al., Biochem. Biophys. Acta 1973, 328:456; Andersson et al., Thromb. Res. 1975, 7:451; Suomela et al., Eur. J. Biochem. 1976, 71:145. In some embodiments the factor IX are factor IX-related polypeptides wherein the ratio between the activity of said factor IX polypeptide and the activity of native human factor IX (wild-type factor IX) is at least about 1.25 when tested in the “chromogenic assay” (see US 20030203845); in other embodiments, the ratio is at least about 2.0; in further embodiments, the ratio is at least about 4.0. Commercially available FIX products (so-called replacement products) are derived from normal pooled plasma or genetically engineered mammalian cell lines. Replacement products are often classified according to final purity, defined as specific activity (international units of clotting factor activity per mg of protein, IU/mg). Intermediate products have relatively low specific activity (<50 IU/mg) because they also contain extraneous plasma proteins, such as fibrinogen, fibronectin and other non-coagulant proteins. High purity (>50 IU/mg) and ultra high purity (>160 IU/mg) contain little or virtual no other plasma proteins other that albumin added as a stabilizer. Non-limiting examples of commercially available Factor IX products (concentrates and preparations) that may be used according to the present invention are, for example, without limitation, BeneFix (rFIX) from AHP/Genetics Institute, Mononine (FIX) from Aventis, Proplex (FIX complex) from Baxter, Bebulin VH (FIX complex) from Baxter, FACTEUR IX-LFB (human plasma-based FIX) both from Laboratoire Francais du Fractionnement et des Biotechnologies (LFB), Immunine (Baxter/Immuno), Octanyne (Octapharma), Octanine F (Octapharma), Mono FIX-VF (CSL), Novact M (Kaketsuken). Factor IX and Factor X proteins, including Factor IX and Factor X variants and derivatives thereof also are known in the art (see, e.g., WO2006127896; EP1728798-A1; and Schuettrumpf et al., Blood, 105 (6): 2316-2323 (2005).

In one embodiment, the combination of a coagulation factor or hemostatic factor and the CPAM agent are the only active agents in the composition or method (or the combination of a CPAM agent and coagulation factor/hemostatic factor and/or second anti-inflammatory agent are the only active agents in the composition/method). In general, any of these agents involved in the methods or compositions described herein can be characterized as being at least substantially isolated or purified.

In some embodiments, the compositions further comprise and/or methods make use of nucleic acids, having sequences encoding a coagulation factor, such as those of NCBI's GenBank, Accession Number CAH72549, AAA88042, AAT85802, P08709, P00740, P12259, P00748, P03951, KFHU5, P00488, AAB31518, AAB31517, AAB31516, NP_{—}000121, NP_{—}000124, P00742, NP_{—}000123, NP_{—}000495, NP_{—}001984, 1205236A, AAB59401, NP_{—}036283, CAI43241, AAH98389, AAH22513, AAB28588, AAI22864, AAI19015, AAH64380, AAI11970, AAI11968, AAC50211, AAA52420, AAB5949, AAA70225, AAA58466, AAV85964, AAB59490, AAA52421, AAA51986, CAI95417, CAI95416, CAI42103, CAI41386, CAI41385, CAI41382, CAI41381, AAI30469, AAH27963, EAX09185, EAX09184, EAX09183, EAX09182, EAX04621, EAX04620, ABJ51930, ABJ51929, ABJ51928, ABJ51927, ABJ51926, ABJ51925, ABJ51924, ABJ51923, ABJ51922, ABJ51921, ABJ51931, ABI26644, BAA20295, ABF69029, ABF69028, ABF69027, ABF69026, ABF69025, ABF69024, ABF69023, ABF69022, ABF69021, ABF69020, ABF69019, ABF69018, ABF69017, ABF69016, ABF69015, ABF69013, ABF69012, ABF69011, ABF69010, ABF69009, ABF69007, ABF69006, ABF69005, CAA48315, CAA48314, ABK19831, ABK19830, ABK19829, ABK19827, ABJ55913, ABJ55912, ABJ55910, ABJ55909, or others known to those skilled in the art, or a homologue or variant thereof, or combinations thereof.

In one embodiment, the method further comprises administering an agent, which promotes or enhances production of a coagulation factor or other hemostatic agent to said subject, which in one embodiment is a nucleic acid encoding said coagulation factor, which in another embodiment encodes Factor VIII.

In some embodiments, the nucleic acid encoding Factor VIII is a vector, which comprises the first 57 base pairs of the Factor VIII heavy chain, for example as described in United States Patent Application Publication Number 20050276787. In some embodiments, the vector will comprise the full-length human factor VIII, for example, as described in U.S. Pat. No. 4,757,006, or in Toole et al., Nature 312: 312-317, 1984; Wood et al., Nature 312: 330-337, 1984; Vehar et al.; Nature 312: 337-342, 1984, WO 87/04187, WO 88/08035 and WO 88/03558, U.S. Pat. No. 4,868,112; EP 0 786 474; WO 86/06101; WO 87/07144, and others known in the art.

In some embodiments, vectors and constructs can be introduced into a cell using standard methodology known to those of skill in the art (e.g., transfection). A number of transfection techniques are generally known in the art (See e.g., Graham et al., Virol., 52:456 [1973], Sambrook et al. supra, Davis et al., supra, and Chu et al., Gene 13:197 [1981]). Particularly suitable transfection methods include calcium phosphate co-precipitation (Graham et al., Virol., 52:456-467 [1973]), direct micro-injection into cultured cells (Capecchi, Cell 22:479-488 [1980]), electroporation (Shigekawa et al., BioTechn., 6:742-751 [1988]), liposome-mediated gene transfer (Mannino et al., BioTechn., 6:682-690 [1988]), lipid-mediated transduction (Feigner et al., Proc. Natl. Acad. Sci. USA 84:7413-7417 [1987]), and nucleic acid delivery using high-velocity microprojectiles (Klein et al., Nature 327:70-73 [1987]).

In some embodiments, Factor VIII expression and activity can be assayed by any means, for example, Western blot analysis, Northern blot analysis, Southern blot analysis, denaturing polyacrylamide gel electrophoresis (e.g. SDS-PAGE), reverse transcriptase-coupled polymerase chain reaction (RT-PCR), enzyme-linked immunosorbent assay (ELISA), radioimmunoassay (RIA), and fluorescent immunoassay (IFA).

In another aspect, the coagulation factor is Factor VII or a variant or derivative thereof. In some embodiments, the Factor VIIa (the terms Factor VII and Factor VIIa are used somewhat interchangeably herein, it being recognized that any suitable form of a coagulation factor can be used/incorporated in the methods/compositions of the invention, active forms being typically used/incorporated) is human Factor VIIa, as disclosed, e.g., in U.S. Pat. No. 4,784,950 (wild-type Factor VII), as SEQ ID NO:1 thereof. As noted already, the term “Factor VII” is intended to encompass Factor VII polypeptides in their uncleaved (zymogen) form, as well as those that have been proteolytically processed to yield their respective bioactive forms, which may be designated Factor VIIa. Typically, human Factor VII is cleaved between residues 152 and 153 to yield Factor VIIa.

In another embodiment, a CPAM agent is combined with a Factor VII analogue or variant (the terms analog, analogue, and variant are used interchangeably herein). A FVII variant can be any variant that differs by one or more insertions, deletions, additions, etc. from wild-type FVII (typically a variant will have at least about 85%, at least about 90%, at least about 95%, at least about 97% or more identity to the wild-type human FVII amino acid sequence and at least substantially retain similar some of the enzymatic activity as human FVII). Non-limiting examples of Factor VII/Factor VIIa (FVIIa) variants include S52A; 560A; FVIIa variants exhibiting increased proteolytic stability as disclosed in U.S. Pat. No. 5,580,560; Factor VII variants as disclosed in PCT/DK02/00189; Factor VII variants exhibiting increased proteolytic stability as disclosed in WO 02/38162; Factor VII variants having a modified Gla-domain and exhibiting an enhanced membrane binding as disclosed in WO 99/20767; and Factor VII variants as disclosed in WO 01/58935; WO 01/83725; WO 02/22776; WO 02/077218; PCT/DK02/00635; Danish patent application PA 2002 01423; Danish patent application PA 2001 01627; WO 02/38162; FVIIa variants with enhanced activity as disclosed in JP 2001061479; and Factor VIIa lacking the Gla domain, (Nicolaisen et al., FEBS Letts. 317:245-249, 1993). Further non-limiting examples of additional modifications include: R152E; S344A; L305V; L305V/M306D/D309S; L3051, L305T, F374P, V158T/M298Q, V158D/E296V/M298Q, K337A, M298Q, V158D/M298Q, L305V/K337A, V158D/E296V/M298Q/L305V, V158D/E296V/M298Q/K337A, V158D/E296V/M298Q/L305V/K337A, K157A, E296V, E296V/M298Q, V158D/E296V, V158D/M298K, and S336G, L305V/K337A, L305V/V158D, L305V/E296V, L305V/M298Q, L305V/V158T, L305V/K337A/V158T, L305V/K337A/M298Q, L305V/K337A/E296V, L305V/K337A/V158D, L305V/V158D/M298Q, L305V/V158D/E296V, L305V/V158T/M298Q, L305V/V158T/E296V, L305V/E296V/M298Q, L305V/V158D/E296V/M298Q, L305V/V158T/E296V/M298Q, L305V/V158T/K337A/M298Q, L305V/V158T/E296V/K337A, L305V/V158D/K337A/M298Q, L305V/V158D/E296V/K337A-FVII, L305V/V158D/E296V/M298Q/K337A, L305V/V158T/E296V/M298Q/K337A, S314E/K316H, S314E/K316Q, S314E/L305V, S314E/K337A, S314E/V158D, S314E/E296V, S314E/M298Q, S314E/V158T, K316H/L305V, K316H/K337A, K316H/V158D, K316H/E296V, K316H/M298Q, K316H/V158T, K316Q/L305V, K316Q/K337A, K316Q/V158D, K316Q/E296V, K316Q/M298Q, K316Q/V158T, S314E/L305V/K337A, S314E/L305V/V158D, S314E/L305V/E296V, S314E/L305V/M298Q, S314E/L305V/V158T, S314E/L305V/K337A/V158T, S314E/L305V/K337A/M298Q, S314E/L305V/K337A/E296V, S314E/L305V/K337A/V158D, S314E/L305V/V158D/M298Q, S314E/L305V/V158D/E296V, S314E/L305V/V158T/M298Q, S314E/L305V/V158T/E296V, S314E/L305V/E296V/M298Q, S314E/L305V/V158D/E296V/M298Q, S314E/L305V/V158T/E296V/M298Q, S314E/L305V/V158T/K337A/M298Q, S314E/L305V/V158T/E296V/K337A, S314E/L305V/V158D/K337A/M298Q, S314E/L305V/V158D/E296V/K337A, S314E/L305V/V158D/E296V/M298Q/K337A, S314E/L305V/V158T/E296V/M298Q/K337A, K316H/L305V/K337A, K316H/L305V/V158D, K316H/L305V/E296V, K316H/L305V/M298Q, K316H/L305V/V158T, K316H/L305V/K337A/V158T, K316H/L305V/K337V/M298Q, K316H/L305V/K337A/E296V, K316H/L305V/K337A/V158D, K316H/L305V/V158D/M298Q, K316H/L305V/V158D/E296V, K316H/L305V/V158T/M298Q, K316H/L305V/V158T/E296V, K316H/L305V/E296V/M298Q, K316H/L305V/V158D/E296V/M298Q, K316H/L305V/V158T/E296V/M298Q, K316H/L305V/V158T/K337A/M298Q, K316H/L305V/V158T/E296V/K337A, K316H/L305V/V158D/K337A/M298Q, K316H/L305V/V158D/E296V/K337A, K316H/L305V/V158D/E296V/M298Q/K337A, K316H/L305V/V158T/E296V/M298Q/K337A, K316Q/L305V/K337A, K316Q/L305V/V158D, K316Q/L305V/E296V, K316Q/L305V/M298Q, K316Q/L305V/V158T, K316Q/L305V/K337A/V158T, K316Q/L305V/K337A/M298Q, K316Q/L305V/K337A/E296V, K316Q/L305V/K337A/V158D, K316Q/L305V/V158D/M298Q, K316Q/L305V/V158D/E296V, K316Q/L305V/V158T/M298Q, K316Q/L305V/V158T/E296V, K316Q/L305V/E296V/M298Q, K316Q/L305V/V158D/E296V/M298Q, K316Q/L305V/V158T/E296V/M298Q, K316Q/L305V/V158T/K337A/M298Q, K316Q/L305V/V158T/E296V/K337A, K316Q/L305V/V158D/K337A/M298Q, K316Q/L305V/V158D/E296V/K337A, K316Q/L305V/V158D/E296V/M298Q/K337A, K316Q/L305V/V158T/E296V/M298Q/K337A, F374Y/K337A, F374Y/V158D, F374Y/E296V, F374Y/M298Q, F374Y/V158T, F374Y/S314E, F374Y/L305V, F374Y/L305V/K337A, F374Y/L305V/V158D, F374Y/L305V/E296V, F374Y/L305V/M298Q, F374Y/L305V/V158T, F374Y/L305V/S314E, F374Y/K337A/S314E, F374Y/K337A/V158T, F374Y/K337A/M298Q, F374Y/K337A/E296V, F374Y/K337A/V158D, F374Y/V158D/S314E, F374Y/V158D/M298Q, F374Y/V158D/E296V, F374Y/V158T/S314E, F374Y/V158T/M298Q, F374Y/V158T/E296V, F374Y/E296V/S314E, F374Y/S314E/M298Q, F374Y/E296V/M298Q, F374Y/L305V/K337A/V158T, F374Y/L305V/K337A/E296V, F374Y/L305V/K337A/M298Q, F374Y/L305V/K337A/V158T, F374Y/L305V/K337A/S314E, F374Y/L305V/V158D/E296V, F374Y/L305V/V158D/M298Q, F374Y/L305V/V158D/S314E, F374Y/L305V/E296V/M298Q, F374Y/L305V/E296V/V158T, F374Y/L305V/E296V/S314E, F374Y/L305V/M298Q/V158T, F374Y/L305V/M298Q/S314E, F374Y/L305V/V158T/S314E, F374Y/K337A/S314E/V158T, F374Y/K337A/S314E/M298Q, F374Y/K337A/S314E/E296V, F374Y/K337A/S314E/V158D, F374Y/K337A/V158T/M298Q, F374Y/K337A/V158T/E296V, F374Y/K337A/M298Q/E296V, F374Y/K337A/M298Q/V158D, F374Y/K337A/E296V/V158D, F374Y/V158D/S314E/M298Q, F374Y/V158D/S314E/E296V, F374Y/V158D/M298Q/E296V, F374Y/V158T/S314E/E296V, F374Y/V158T/S314E/M298Q, F374Y/V158T/M298Q/E296V, F374Y/E296V/S314E/M298Q, F374Y/L305V/M298Q/K337A/S314E, F374Y/L305V/E296V/K337A/S314E, F374Y/E296V/M298Q/K337A/S314E, F374Y/L305V/E296V/M298Q/K337A, F374Y/L305V/E296V/M298Q/S314E, F374Y/V158D/E296V/M298Q/K337A, F374Y/V158D/E296V/M298Q/S314E, F374Y/L305V/V158D/K337A/S314E, F374Y/V158D/M298Q/K337A/S314E, F374Y/V158D/E296V/K337A/S314E, F374Y/L305V/V158D/E296V/M298Q, F374Y/L305V/V158D/M298Q/K337A, F374Y/L305V/V158D/E296V/K337A, F374Y/L305V/V158D/M298Q/S314E, F374Y/L305V/V158D/E296V/S314E, F374Y/V158T/E296V/M298Q/K337A, F374Y/V158T/E296V/M298Q/S314E, F374Y/L305V/V158T/K337A/S314E, F374Y/V158T/M298Q/K337A/S314E, F374Y/V158T/E296V/K337A/S314E, F374Y/L305V/V158T/E296V/M298Q, F374Y/L305V/V158T/M298Q/K337A, F374Y/L305V/V158T/E296V/K337A, F374Y/L305V/V158T/M298Q/S314E, F374Y/L305V/V158T/E296V/S314E, F374Y/E296V/M298Q/K337A/V158T/S314E, F374Y/V158D/E296V/M298Q/K337A/S314E, F374Y/L305V/V158D/E296V/M298Q/S314E, F374Y/L305V/E296V/M298Q/V158T/S314E, F374Y/L305V/E296V/M298Q/K337A/V158T, F374Y/L305V/E296V/K337A/V158T/S314E, F374Y/L305V/M298Q/K337A/V158T/S314E, F374Y/L305V/V158D/E296V/M298Q/K337A, F374Y/L305V/V158D/E296V/K337A/S314E, F374Y/L305V/V158D/M298Q/K337A/S314E, F374Y/L305V/E296V/M298Q/K337A/V158T/S314E,

F374Y/L305V/V158D/E296V/M298Q/K337A/S314E; R152E, S344A; P11Q/K33E, T106N, V253N, R290N/A292T, G291N, R315N/V317T, K143N/R315N/V317T; FVII having substitutions, additions or deletions in the amino acid sequence from T233 to N240; and FVII having substitutions, additions or deletions in the amino acid sequence from R304 to C329.

Non-limiting examples of Factor VIIa derivatives include wild-type or analogue Factor VIIa polypeptides to which polyethylene glycol (PEG) polymers, acyl groups, phosphate or sulphate groups, and the like have been attached. Factor VIIa derivatives may also comprise Factor VII polypeptides containing N-linked or O-linked oligosaccharides that have been chemically or enzymatically modified, or Factor VIIa (wild type or analogue) polypeptides that have been subjected to proteolytic cleavage. Non-limiting examples of Factor VII derivatives includes GlycoPegylated FVII derivatives as disclosed in WO 03/31464 and US Patent applications US 20040043446, US 20040063911, US 20040142856, US 20040137557, and US 20040132640 (Neose Technologies, Inc.); FVII conjugates as disclosed in WO 02/077218, US 20030044908 (Novo Nordisk A/S; WO 01/04287, US patent application 20030165996, WO 01/58935, WO 03/93465 (Maxygen ApS) and WO 02/02764, US patent application 20030211094 (University of Minnesota).

In one embodiment, the CPAM agent abrogates or diminishes C5a expression, C5a binding to a cognate receptor, C5a receptor signaling, or a combination thereof.

In one embodiment, this invention provides a pharmaceutical composition comprising an agent, which abrogates or diminishes activation of a complement pathway and a coagulation factor or other hemostatic agent.

In one embodiment, this invention provides a pharmaceutical composition comprising an agent, which abrogates or diminishes activation of a complement pathway and a nucleic acid encoding a coagulation factor or other hemostatic agent.

In one embodiment, the CPAM agent is a biologic, which in one embodiment is an antibody, or in another embodiment is an oligopeptide. In another embodiment, the coagulation factor is Factor VIII.

In another embodiment, the CPAM agent abrogates or diminishes C5a expression, C5a binding to a cognate receptor, C5a receptor signaling, or a combination thereof.

In another embodiment, the pharmaceutical composition is formulated for oral, intravenous, subcutaneous, in-joint (local) or topical administration, or in another embodiment, the pharmaceutical composition is formulated as an injectable formulation.

This invention further provides, in some embodiments, compositions of this invention, and methods of use thereof, wherein the compositions or methods further comprise one or more second anti-inflammatory therapeutic agent(s) or procedures, examples which include, but are not limited to, synthetic and non-synthetic corticosteroid agents, nonsteroidal anti-inflammatory drugs, antirheumatics, immunoregulators, immunosuppressors, articular function augmenters, interleukin production inhibitors, or combinations thereof. Specific examples of corticosteroid agents include, but are not limited to dexamethasone, hydrocortisone, triamcinolone, betamethasone, predonisolone, methylpredonisolone, halopredone, beclomethasone and the like. Additional examples of such agents are described in, e.g., Romas et al., Rheum Dis Clin North Am. 2006 November; 32 (4):759-73; Zhonghua et al., 2006 November 21; 86 (43):3055-8; and Katz et al., Curr Opin Rheumatol. 2007 March; 19 (2):106-110.

Further specific examples of non-steroidal anti inflammatory agents include, but are not limited to diclofenac, indomethacin, ibuprofen, ketoprofen, aspirin, diflunisal, fulfenamic acid, floctafenine, tolfenamic acid, sulindac, fenbufen, salicylic acid, acemetacin, proglumetacin, nabumetone, protizinic acid, thiaprofen, oxaprozin, loxoprofen, alminoprofen, zaltoprofen, flurbiprofen, flurbiprofen and the like. The skilled practitioner will take into consideration health of the patient, age, etc., when considering any combination of a CPAM agent with an anti-inflammatory drug, particularly given known health risks associated with these compounds.

In one embodiment, the compositions of present invention can further comprise of at least one pyrazolyl benzenesulfonamide compound, e.g., as set forth in U.S. Pat. No. 5,756,529 and U.S. Pat. No. 5,466,823. In some embodiments, the compositions of the invention can further comprise a diaryl substituted pyrazole useful for treatment of inflammation and/or pain. In some embodiments, the compositions of the invention can further comprise therapeutic amounts of any of the class of diaryl substituted pyrazoles their isomers, analogs and/or metabolites. In some embodiments, these compounds reduce inflammation and/or pain primarily via inhibition of cyclooxygenase-2 (COX-2).

In some embodiments, the compositions further comprise a non-steroidal agent that reduces inflammation and/or pain, such as, for example, celecoxib, rofecoxib and the like.

In some embodiments, the compositions further comprise axetil, piroxicam, tenoxicam, ampiroxicam, meloxicam, D-penicillamine, bucillamine, gold sodium thiomalate, auranofin, lobenzarit, salazosulfapyridine, methotrexate, cyclophosphamide, azathioprine, mizoribine, cyclosporin and the like.

In some embodiments, the compositions further comprise opioids and other analgesics, including narcotic analgesics, Mu receptor antagonists, Kappa receptor antagonists, non-narcotic (i.e. non-addictive) analgesics, monoamine uptake inhibitors, adenosine regulating agents, cannabinoid derivatives, Substance P antagonists, neurokinin-1 receptor antagonists and sodium channel blockers, among others. In some embodiments, the compositions further comprise aceclofenac, acemetacin, e-acetamidocaproic acid, acetaminophen, acetaminosalol, acetanilide, acetylsalicylic acid (aspirin), S-adenosylmethionine, alclofenac, alfentanil, allylprodine, alminoprofen, aloxiprin, alphaprodine, aluminum bis(acetylsalicylate), amfenac, aminochlorthenoxazin, 3-amino-4-hydroxybutyric acid, 2-amino-4-picoline, aminopropylon, aminopyrine, amixetrine, ammonium salicylate, ampiroxicam, amtolmetin guacil, anileridine, antipyrine, antipyrine salicylate, antrafenine, apazone, bendazac, benorylate, benoxaprofen, benzpiperylon, benzydamine, benzylmorphine, bermoprofen, bezitrarnide, .alpha.-bisabolol, bromfenac, .rho.-bromoacetanilide, 5-bromosalicylic acid acetate, bromosaligenin, bucetin, bucloxic acid, bucolome, bufexamac, bumadizon, buprenorphine, butacetin, butibufen, butophanol, calcium acetylsalicylate, carbamazepine, carbiphene, carprofen, carsalam, chlorobutanol, chlorthenoxazin, choline salicylate, cinchophen, cinmetacin, ciramadol, clidanac, clometacin, clonitazene, clonixin, clopirac, clove, codeine, codeine methyl bromide, codeine phosphate, codeine sulfate, cropropamide, crotethamide, desomorphine, dexoxadrol, dextromoramide, dezocine, diampromide, diclofenac sodium, difenamizole, difenpiramide, diflunisal, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, epirizole, eptazocine, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, flufenamic acid, flunoxaprofen, fluoresone, flupirtine, fluproquazone, flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, isofezolac, isoladol, isomethadone, isonixin, isoxepac, isoxicam, ketobemidone, ketoprofen, ketorolac, p-lactophenetide, lefetamine, levorphanol, lofentanil, lonazolac, lomoxicam, loxoprofen, lysine acetylsalicylate, magnesium acetylsalicylate, meclofenamic acid, mefenamic acid, meperidine, meptazinol, mesalamine, metazocine, methadone hydrochloride, methotrimeprazine, metiazinic acid, metofoline, metopon, mofebutazone, mofezolac, morazone, morphine, morphine hydrochloride, morphine sulfate, morpholine salicylate, myrophine, nabumetone, nalbuphine, 1-naphthyl salicylate, naproxen, narceine, nefopam, nicomorphine, nifenazone, niflurmic acid, nimesulide, 5′-nitro-2′-propoxyacetanilide, norlevorphanol, norrmethadone, normorphine, norpipanone, olsalazine, opium, oxaceprol, oxametacine, oxaprozin, oxycodone, oxymorphone, oxyphenbutazone, papaveretum, paranyline, parsalmide, pentazocine, perisoxal, phenacetin, phenadoxone, phenazocine, phenazopyridine hydrochloride, phenocoll, phenoperidine, phenopyrazone, phenyl acetylsalicylate, phenylbutazone, phenyl salicylate, phenyramidol, piketoprofen, piminodine, pipebuzone, piperylone, piprofen, pirazolac, piritramide, piroxicam, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamide, salicin, salicylamide, salicylamide o-acetic acid, salicylsulfuric acid, salsalte, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tolfenamic acid, tolmetin, tramadol, tropesin, viminol, xenbucin, ximoprofen, zaltoprofen and zomepirac (see The Merck Index, 12th Edition, Therapeutic Category and Biological Activity Index, ed. S. Budavari (1996), pp. Ther-2 to Ther-3 and Ther-12 (Analgesic (Dental), Analgesic (Narcotic), Analgesic (Non-narcotic), Anti-inflammatory (Nonsteroidal)).

The compounds and pharmaceutical formulations according to the invention may be used in combination with or include one or more other therapeutic agents, for example selected from anti-inflammatory agents, anticholinergic agents (particularly an M1, M2, M1/M2 or M3 receptor antagonist), β2-adrenoreceptor agonists, antiinfective agents (e.g. antibiotics, antivirals), antihistamines, as described herein, or any combinations thereof. This invention provides, in some embodiments, a combination of an agent, which inhibits or interferes with a complement pathway together with one or more other therapeutically active agents, for example a coagulation factor, and/or optionally an anti-inflammatory agent (for example a corticosteroid or an NSAID), an anticholinergic agent, β2-adrenoreceptor agonist, an antiinfective agent (e.g. an antibiotic or an antiviral), or an antihistamine, or a corticosteroid, or an anticholinergic, or a PDE-4 inhibitor, or any combination thereof.

It will be clear to a person skilled in the art that, where appropriate, the therapeutic ingredient(s) may be used in the form of salts, (e.g. as alkali metal or amine salts or as acid addition salts), or prodrugs, or as esters (e.g. lower alkyl esters), or as solvates (e.g. hydrates) to optimise the activity and/or stability and/or physical characteristics (e.g. solubility) of the therapeutic ingredient. It will be clear also that where appropriate, the therapeutic ingredients may be used in optically pure form.

Examples of β2-adrenoreceptor agonists include salmeterol (which may be a racemate or a single enantiomer, such as the R-enantiomer), salbutamol, formoterol, salmefamol, fenoterol or terbutaline and salts thereof, for example the xinafoate salt of salmeterol, the sulphate salt or free base of salbutamol or the fumarate salt of formoterol. Long-acting β2-adrenoreceptor agonists are preferred, especially those having a therapeutic effect over a 24 hour period, such as salmeterol or formoterol.

In some embodiments, the β2-adrenoreceptor agonists include those described in WO02/66422A, WO02/270490, WO02/076933, WO03/024439, WO03/072539, WO 03/091204, WO04/016578, WO04/022547, WO04/037807, WO04/037773, WO04/037768, WO04/039762, WO04/039766, WO01/42193 and WO03/042160, whose disclosures are incorporated by reference herein.

In some embodiments, the β2-adrenoreceptor agonists are: 3-(4-{[6-({(2R)-2-hydroxy-2-[4-hydroxy-3-(hydroxymethyl)phenyl]ethyl}amino) hexyl]oxy}butyl)benzenesulfonamide; 3-(3-{[7-({(2R)-2-hydroxy-2-[4-hydroxy-3-hydroxymethyl)phenyl]ethyl}-amino)heptyl]oxy}propyl)benzenesulfonamide; 4-{(1R)-2-[(6-{2-[(2,6-dichlorobenzyl)oxy]ethoxy}hexyl)amino]-1-hydroxyethyl}-2(hydroxymethyl)phenol;

4-{(1R)-2-[(6-{4-[3-(cyclopentylsulfonyl)phenyl]butoxy}hexyl)amino]-1 hydroxyethyl}-2-(hydroxymethyl)phenol; N-[2-hydroxyl-5-[(1R)-1-hydroxy-2-[[2-4-[[(2R)-2-hydroxy-2-phenylethyl]amino]phenyl]ethyl]amino]ethyl]phenyl]foramide, N-2 {2-[4-(3-phenyl-4-methoxyphenyl)aminophenyl]ethyl}-2-hydroxy-2-(8-hydroxy-2(1H)-quinolinon-5-yl)ethylamine, or a combination thereof

In some embodiments, the corticosteroids may comprise include methyl prednisolone, prednisolone, dexamethasone, fluticasone propionate, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl)ester, 6α,9α-difluoro-11β-hydroxy-16α-methyl-17α-(1-methylcylopropylcarbonyl)oxy-3-oxo-androsta-1,4-diene-17β-carbothioic acid S-fluoromethyl ester, 6α,9α-difluoro-11β-hydroxy-16α-methyl-3-oxo-17α-(2,2,3,3-tetramethylcyclopropylcarbonyl)oxy-androsta-1,4-diene-17β-carboxylic acid cyanomethyl ester, beclomethasone esters (such as the 17-propionate ester or the 17,21-dipropionate ester), budesonide, flunisolide, mometasone esters (such as the furoate ester), triamcinolone acetonide, rofleponide, ciclesonide, (16α,17-[[(R)-cyclohexylmethylene]bis(oxy)]-11β,21-dihydroxy-pregna-1,4-diene-3,20-dione), butixocort propionate, RPR-106541, and ST-126. Preferred corticosteroids include fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester and 6α,9α-difluoro-17α-[(2-furanylcarbonyl)oxy]-11β-hydroxy-16α-methyl-3-oxo-androsta-1,4-diene-17β-carbothioic acid S-fluoromethyl ester, more preferably 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

In some embodiments, the compositions comprise/methods make use of a non-steroidal compound having glucocorticoid agonism that may possess selectivity for transrepression over transactivation, such as, for example, compounds disclosed in WO03/082827, WO01/10143, WO98/54159, WO04/005229, WO04/009016, WO04/009017, WO04/018429, WO03/104195, WO03/082787, WO03/082280, WO03/059899, WO03/101932, WO02/02565, WO01/16128, WO00/66590, WO03/086294, WO04/026248, WO03/061651, WO03/08277, fully incorporated by reference herein.

In some embodiments, the compositions comprise/methods make use of a non-steroidal anti-inflammatory drugs (NSAID's) such as sodium cromoglycate, nedocromil sodium, phosphodiesterase (PDE) inhibitors (for example, theophylline, PDE4 inhibitors or mixed PDE3/PDE4 inhibitors), leukotriene antagonists, inhibitors of leukotriene synthesis (for example, montelukast), iNOS inhibitors, tryptase and elastase inhibitors, beta-2 integrin antagonists and adenosine receptor agonists or antagonists (for example, adenosine 2a agonists), cytokine antagonists (for example, chemokine antagonists, such as a CCR3 antagonist) or inhibitors of cytokine synthesis, or 5-lipoxygenase inhibitors. Suitable other β2-adrenoreceptor agonists include salmeterol (for example, as the xinafoate), salbutamol (for example, as the sulphate or the free base), formoterol (for example, as the fumarate), fenoterol or terbutaline and salts thereof. An iNOS (inducible nitric oxide synthase inhibitor) is preferably for oral administration. Suitable iNOS inhibitors include those disclosed in WO93/13055, WO98/30537, WO02/50021, WO95/34534 and WO99/62875. Suitable CCR3 inhibitors include those disclosed in WO02/26722.

In some embodiments, the compositions comprise/methods make use of a phosphodiesterase 4 (PDE4) inhibitor or a mixed PDE3/PDE4 inhibitor, for example, cis 4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol].

In some embodiments, the compositions comprise/methods make use of compounds set out in U.S. Pat. No. 5,552,438; that of Hofgen, N. et al. 15th EFMC Int. Symp. Med. Chem. (September 6-10, Edinburgh) 1998, Abst. P. 98; CAS reference No. 247584020-9); a 9-benzyladenine derivative nominated NCS-613 (INSERM); D-4418 from Chiroscience and Schering-Plough; a benzodiazepine PDE4 inhibitor identified as C1-1018 (PD-168787) and attributed to Pfizer; a benzodioxole derivative disclosed by Kyowa Hakko in WO99/16766; K-34 from Kyowa Hakko; V-11294A from Napp (Landells, L. J. et al. Eur Resp J [Annu Cong Eur Resp Soc (September 19-23, Geneva) 1998] 1998, 12 (Suppl. 28): Abst P2393); roflumilast (CAS reference No 162401-32-3) a pthalazinone (WO99/47505, the disclosure of which is hereby incorporated by reference) from Byk-Gulden; Pumafentrine, (−)-p-[(4aR*,10bS*)-9-ethoxy-1,2,3,4,4a,10b-hexahydro-8-methoxy-2 methylbenzo [c] [1,6] naphthyridin-6-yl]-N,N-diisopropylbenzamide which is a mixed PDE3/PDE4 inhibitor which has been prepared and published on by Byk-Gulden, now Altana; arofylline under development by Almirall-Prodesfarma; VM554/UM565 from Vernalis; or T-440 (Tanabe Seiyaku; Fuji, K. et al. J Pharmacol Exp Ther, 1998, 284 (1): 162), and T2585. Other possible PDE-4 and mixed PDE3/PDE4 inhibitors include those listed in WO01/13953, the disclosure of which is hereby incorporated by reference. In some embodiments, the compositions comprise/methods make use of any combination of agents as herein described.

In some embodiments, the compositions comprise/methods make use of anticholinergic agents, such as alkaloids of the belladonna plants as illustrated by the likes of atropine, scopolamine, homatropine, hyoscyamine; these compounds are normally administered as a salt, being tertiary amines. These drugs, particularly the salt forms, are readily available from a number of commercial sources or can be made or prepared from literature for example: Atropine—CAS-51-55-8 or CAS-51-48-1 (anhydrous form), atropine sulfate—CAS-5908-99-6; atropine oxide—CAS-4438-22-6 or its HCl salt—CAS-4574-60-1 and methylatropine nitrate—CAS-52-88-0; Homatropine—CAS-87-00-3, hydrobromide salt—CAS-51-56-9, methylbromide salt—CAS-80-49-9; Hyoscyamine (d, 1)—CAS-101-31-5, hydrobromide salt—CAS-306-03-6 and sulfate salt—CAS-6835-16-1; and Scopolamine—CAS-51-34-3, hydrobromide salt—CAS-6533-68-2, methylbromide salt—CAS-155-41-9.

In some embodiments, the compositions comprise/methods make use of anticholinergics such as, but not limited to, ipratropium (e.g. as the bromide), sold under the name Atrovent, oxitropium (e.g. as the bromide) and tiotropium (e.g. as the bromide) (CAS-139404-48-1). Also of interest are: methantheline (CAS-53-46-3), propantheline bromide (CAS-50-34-9), anisotropine methyl bromide or Valpin 50 (CAS-80-50-2), clidinium bromide (Quarzan, CAS-3485-62-9), copyrrolate (Robinul), isopropamide iodide (CAS-71-81-8), mepenzolate bromide (U.S. Pat. No. 2,918,408), tridihexethyl chloride (Pathilone, CAS-4310-35-4), and hexocyclium methylsulfate (Tral, CAS-115-63-9). See also cyclopentolate hydrochloride (CAS-5870-29-1), tropicamide (CAS-1508-75-4), trihexyphenidyl hydrochloride (CAS-144-11-6), pirenzepine (CAS-29868-97-1), telenzepine (CAS-80880-90-9), AF-DX 116, or methoctramine, and the compounds disclosed in WO 01/04118, the disclosure of which is hereby incorporated by reference.

In some embodiments, the compositions comprise/methods make use of antihistamines (also referred to as H1-receptor antagonists), which may include any one or more of the numerous antagonists known which inhibit H1-receptors, and are safe for human use. In some embodiments, such compounds comprise ethanolamines, ethylenediamines, and alkylamines, such as, but not limited to carbinoxamine maleate, clemastine fumarate, diphenylhydramine hydrochloride, and dimenhydrinate, pyrilamine maleate, tripelennamine HCl, and tripelennamine citrate, chloropheniramine and its salts such as the maleate salt, and acrivastine, hydroxyzine HCl, hydroxyzine pamoate, cyclizine HCl, cyclizine lactate, meclizine HCl, and cetirizine HCl, Astemizole, levocabastine HCl, loratadine or its descarboethoxy analogue, and terfenadine and fexofenadine hydrochloride or another pharmaceutically acceptable salt, azelastine hydrochloride, or any combination of agents as herein described.

The combinations referred to above may conveniently be presented for use in the form of a pharmaceutical formulation and thus pharmaceutical formulations comprising a combination as defined above together with a physiologically acceptable diluent or carrier represent a further aspect of the invention.

The compound of the invention and/or any secondary agents (e.g., hemostatic and/or coagulation factors) can be formulated with any number of suitable carriers, diluents, excipients, and the like (See e.g., Powell et al. “Compendium of excipients for parenteral formulations” PDA J Pharm Sci Technol. 52:238-311 (1998)—the terms “vehicle” and “carrier” can be used to refer to collectively all and, by reference, independently each such type of agent throughout the description of the invention) and/or functionality enhancers (e.g., stabilizers, surfactants, wetting agents, emulsifying agents, preservatives, fillers, salt(s), solubilizers, detergents, anti-aggregating agents (e.g., anti-aggregating amino acid formulations) dispersion media, isotonic agents, tissue fixatives, chelating agents, buffers, antibacterial agents, antioxidants, colorants, flavoring agents, absorption delaying agents, controlled release agents, etc.) appropriate for the indicated route of administration (and contemplated storage, etc.). Such additional ingredients generally are selected so as to not adversely affect the overall stability of the pharmaceutical formulation according to the invention. Suitable carriers, diluents, adjuvants, as well as functionality enhancers and modes of administration/formulation of such compositions are well known in the pharmaceutical arts. See, e.g., Remington: The Science and Practice of Pharmacy, 19th edition, 1995. See also, e.g., Berge et al., J. Pharm. Sci., 6661), 1-19 (1977); Wang and Hanson, J. Parenteral. Sci. Tech: 42, S4-S6 (1988), U.S. Pat. Nos. 6,165,779 and 6,225,289. Additional relevant principles, methods, and agents are described in, e.g., Urquhart et al., Lancet, 16, 367 (1980), Lieberman et al., PHARMACEUTICAL DOSAGE FORMS-DISPERSE SYSTEMS (2nd ed., vol. 3,1998); Ansel et al., PHARMACEUTICAL DOSAGE FORMS & DRUG DELIVERY SYSTEMS (7th ed. 2000); Martindale, THE EXTRA PHARMACOPEIA (31st edition), Remington's PHARMACEUTICAL SCIENCES (16th-20th editions); The Pharmacological Basis Of Therapeutics, Goodman and Gilman, Eds. (9th ed.—1996); Wilson and Gisvolds' TEXTBOOK OF ORGANIC MEDICINAL AND PHARMACEUTICAL CHEMISTRY, Delgado and Remers, Eds. (10th ed. -1998), and U.S. Pat. Nos. 5,708,025 and 5,994,106. Principles of formulating pharmaceutically acceptable compositions also are described in, e.g., Platt, Clin. Lab Med., 7:289-99 (1987), Aulton, PHARMACEUTICS: THE SCIENCE OF DOSAGE FORM DESIGN, Churchill Livingstone (New York) (1988), EXTEMPORANEOUS ORAL LIQUID DOSAGE PREPARATIONS, CSHP (1998), and “Drug Dosage,” J. Kans. Med. Soc., 70 (I), 30-32 (1969).

Compositions of the invention can be formulated in any suitable form, such as liquid, semi-solid and solid dosage forms, such as liquid solutions (e.g., injectable and infusible solutions), dispersions or suspensions, and the like. The optimal form for any composition depends on the intended mode of administration, the nature of the composition or combination, and therapeutic application or other intended use. A typical mode for delivery for a composition of the invention is by parenteral administration (e.g., intravenous administration). In one aspect, a composition of the invention is administered to a human patient by intravenous infusion or injection. Injection may be accomplished using, e.g., a dual barrel syringe or other suitable device. Examples of potentially suitable delivery devices are described in, e.g., U.S. Pat. No. 5,104,375, No. 4,359,049, No. 4,631,055, and U.S. Pat. No. 4,874,368. Other of suitable delivery devices include pipettes.

Pharmaceutically acceptable compositions typically are sterile, dissolve sufficient amounts of the compound of the invention (and any present secondary agents), are stable under conditions for manufacture and storage, and not harmful to the subject for the proposed application (or at least not harmful to a number, usually a substantial majority (at least about 70%, 80%, 85%, 90%, 95%, 97%, 98%, 99%, etc.), of similar subjects as may be determined by, e.g., clinical trials). A composition so provided by the invention (and/or used in the various methods described elsewhere herein) can be subjected to conventional pharmaceutical operations such as sterilization, purification, etc. (such that the active ingredients thereof can be considered at least substantially isolated or isolated).

In one embodiment of the invention the pharmaceutical formulation is an aqueous formulation, i.e. formulation comprising water. Such formulation is typically a solution or a suspension. In a further embodiment of the invention the pharmaceutical formulation is an aqueous solution. The term “aqueous formulation” is defined as a formulation comprising at least 50% w/w water Likewise, the term “aqueous solution” is defined as a solution comprising at least 50% w/w water, and the term “aqueous suspension” is defined as a suspension comprising at least 50% w/w water.

In another embodiment the pharmaceutical formulation is a freeze-dried formulation, whereto the physician or the patient adds solvents and/or diluents prior to use.

An injectable pharmaceutical product typically is considered “acceptable for therapeutic application” if it is sterile, substantially pyrogen-free, and has no medically unacceptable effects. For example, the product should not produce a medically unacceptable immunological reaction when injected into a human subject. Medically unacceptable effects can be determined by the skilled person in the field of medicine. By purifying the components of the compositions, formulating such components according to the principles described herein and known in the art, and using standard testing procedures, compositions meeting these and/or the other characteristics described herein can be obtained without undue experimentation or effort.

The individual compounds of combination therapies provided by this invention may be administered either sequentially or simultaneously in separate or combined pharmaceutical formulations. Appropriate doses of known therapeutic agents will be readily appreciated or determinable with routine experimentation by those skilled in the art.

In one embodiment, the agent is administered as part of a pharmaceutical composition, which in one embodiment, is formulated for oral, intravenous or topical administration or in another embodiment is formulated as an injectable formulation. It is to be understood that any route of administration, and any formulation for a particular route of administration of the compositions and regarding the methods of this invention is contemplated and comprises this invention.

In some embodiments, the compositions and their use in this invention allow for the incorporation of excipients, which in some embodiments, are solids or liquids or both. Compositions of the invention containing excipients can be prepared by any technique of pharmacy that comprises admixing the excipients with the described agents.

Compositions suitable for buccal or sublingual administration include, for example, lozenges comprising the selective described agents in a flavored base, such as sucrose and acacia or tragacanth, and pastilles comprising the agent in an inert base such as gelatin and glycerin or sucrose and acacia.

Liquid dosage forms for oral administration include pharmaceutically acceptable suspensions, syrups, and elixirs containing inert diluents commonly used in the art, such as water. Such compositions may also comprise, for example, wetting agents, emulsifying and suspending agents, and sweetening, flavoring, and perfuming agents.

Solid unit dosage forms for oral administration contain the described agent, including in nanoparticulate form, together with excipients and are most conveniently formulated as tablets or capsules. Non-limiting examples follow of excipients that can be used to prepare pharmaceutical compositions of the invention.

Compositions of the invention optionally comprise one or more pharmaceutically acceptable diluents as excipients. Suitable diluents illustratively include, either individually or in combination, lactose, including anhydrous lactose and lactose monohydrate; starches, including directly compressible starch and hydrolyzed starches (e.g., Celutab™ and Emdex™); mannitol; sorbitol; xylitol; dextrose (e.g., Cerelose™ 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of .alpha.- and amorphous cellulose (e.g., Rexcel™) and powdered cellulose; calcium carbonate; glycine; bentonite; polyvinylpyrrolidone; and the like. Such diluents, if present, constitute in total about 5% to about 99%, preferably about 10% to about 85%, and more preferably about 20% to about 80%, of the total weight of the composition. The diluent or diluents selected preferably exhibit suitable flow properties and, where tablets are desired, compressibility.

Lactose and microcrystalline cellulose, either individually or in combination, are preferred diluents. Both diluents are chemically compatible with celecoxib. The use of extragranular microcrystalline cellulose (that is, microcrystalline cellulose added to a wet granulated composition after a drying step) can be used to improve hardness (for tablets) and/or disintegration time. Lactose, especially lactose monohydrate, is particularly preferred. Lactose typically provides compositions having suitable release rates of celecoxib, stability, pre-compression flowability, and/or drying properties at a relatively low diluent cost. It provides a high density substrate that aids densification during granulation (where wet granulation is employed) and therefore improves blend flow properties.

Compositions of the invention optionally comprise one or more pharmaceutically acceptable disintegrants as excipients, particularly for tablet formulations. Suitable disintegrants include, either individually or in combination, starches, including sodium starch glycolate (e.g., Explotab™ of PenWest) and pregelatinized corn starches (e.g., National™ 1551, National™ 1550, and Colocorn™ 1500), clays (e.g., Veegum™ HV), celluloses such as purified cellulose, microcrystalline cellulose, methylcellulose, carboxymethylcellulose and sodium carboxymethylcellulose, croscarmellose sodium (e.g., Ac-Di-Sol™ of FMC), alginates, crospovidone, and gums such as agar, guar, locust bean, karaya, pectin and tragacanth gums.

Disintegrants may be added at any suitable step during the preparation of the composition, particularly prior to granulation or during a lubrication step prior to compression. Such disintegrants, if present, constitute in total about 0.2% to about 30%, preferably about 0.2% to about 10%, and more preferably about 0.2% to about 5%, of the total weight of the composition.

Croscarmellose sodium is a preferred disintegrant for tablet or capsule disintegration, and, if present, preferably constitutes about 0.2% to about 10%, more preferably about 0.2% to about 7%, and still more preferably about 0.2% to about 5%, of the total weight of the composition. Croscarmellose sodium confers superior intragranular disintegration capabilities to granulated compositions of the present invention.

Compositions of the invention optionally comprise one or more pharmaceutically acceptable binding agents or adhesives as excipients, particularly for tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; sucrose; gelatin; glucose; starches such as, but not limited to, pregelatinized starches (e.g., National™ 1511 and National™ 1500); celluloses such as, but not limited to, methylcellulose and carmellose sodium (e.g., Tylose™); alginic acid and salts of alginic acid; magnesium aluminum silicate; PEG; guar gum; polysaccharide acids; bentonites; povidone, for example povidone K-15, K-30 and K-29/32; polymethacrylates; HPMC; hydroxypropylcellulose (e.g., Klucel.); and ethylcellulose (e.g., Ethocel™). Such binding agents and/or adhesives, if present, constitute in total about 0.5% to about 25%, preferably about 0.75% to about 15%, and more preferably about 1% to about 10%, of the total weight of the composition.

Compositions of the invention optionally comprise one or more pharmaceutically acceptable wetting agents as excipients. Such wetting agents are preferably selected to maintain the described agents in close association with water, a condition that is believed to improve bioavailability of the composition.

Non-limiting examples of surfactants that can be used as wetting agents in compositions of the invention include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol™ of Gattefosse), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefosse), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total about 0.25% to about 15%, preferably about 0.4% to about 10%, and more preferably about 0.5% to about 5%, of the total weight of the composition.

In some embodiments, wetting agents that are anionic surfactants are utilized. Sodium lauryl sulfate is n embodiment of a wetting agent. Sodium lauryl sulfate, if present, constitutes about 0.25% to about 7%, or in some embodiments, about 0.4% to about 4%, or in some embodiments, about 0.5% to about 2%, of the total weight of the composition.

Compositions of the invention optionally comprise one or more pharmaceutically acceptable lubricants (including anti-adherents and/or glidants) as excipients. Suitable lubricants include, either individually or in combination, glyceryl behapate (e.g., Compritol™ 888); stearic acid and salts thereof, including magnesium, calcium and sodium stearates; hydrogenated vegetable oils (e.g., Sterotex™); colloidal silica; talc; waxes; boric acid; sodium benzoate; sodium acetate; sodium fumarate; sodium chloride; DL-leucine; PEG (e.g., Carbowax™ 4000 and Carbowax™ 6000); sodium oleate; sodium lauryl sulfate; and magnesium lauryl sulfate. In some embodiments, such lubricants, if present, constitute in total about 0.1% to about 10%, or in some embodiments, 0.2% to about 8%, or in some embodiments, 0.25% to about 5%, of the total weight of the composition.

Magnesium stearate is a preferred lubricant used, for example, to reduce friction between the equipment and granulated mixture during compression of tablet formulations.

Suitable anti-adherents include talc, cornstarch, DL-leucine, sodium lauryl sulfate and metallic stearates. Talc is a preferred anti-adherent or glidant used, for example, to reduce formulation sticking to equipment surfaces and also to reduce static in the blend. Talc, if present, constitutes, in some embodiments, about 0.1% to about 10%, or in some embodiments, 0.25% to about 5%, or in some embodiments, about 0.5% to about 2%, of the total weight of the composition.

Other excipients such as colorants, flavors and sweeteners are known in the pharmaceutical art and can be used in compositions of the present invention. Tablets can be coated, for example with an enteric coating, or uncoated. Compositions of the invention can further comprise, for example, buffering agents.

Optionally, one or more effervescent agents can be used as disintegrants and/or to enhance organoleptic properties of compositions of the invention. When present in compositions of the invention to promote dosage form disintegration, one or more effervescent agents in some embodiments, are present in a total amount of about 30% to about 75%, or in some embodiments, 45% to about 70%, for example about 60%, by weight of the composition.

In one embodiment of the invention, the composition is in the form of unit dose capsules or tablets and comprises a partially or wholly nanoparticulate agents as herein described, in a desired amount together with one or more excipients such as pharmaceutically acceptable diluents, disintegrants, binding agents, wetting agents and lubricants. In some embodiments, the composition comprises one or more excipients selected such as lactose (most preferably lactose monohydrate), sodium lauryl sulfate, polyvinylpyrrolidone, croscarmellose sodium, magnesium stearate and microcrystalline cellulose. In some embodiments, the composition comprises lactose monohydrate and croscarmellose sodium. In some embodiments, such a composition further comprises one or more of the carrier materials sodium lauryl sulfate, magnesium stearate and microcrystalline cellulose.

Excipients for capsule and tablet compositions of the invention are selected, in some embodiments, to provide a disintegration time of less than about 30 minutes, or in some embodiments, about 25 minutes or less, or in some embodiments, about 20 minutes or less, or in some embodiments, about 15 minutes or less, in a standard disintegration assay.

Illustratively for tablet formulations, a complete blend of ingredients in an amount sufficient to make a uniform batch of tablets is subjected to tableting in a conventional production scale tableting machine at normal compression pressure (for example, applying a force of about 1 kN to about 50 kN in a typical tableting die). Any tablet hardness convenient with respect to handling, manufacture, storage and ingestion can be obtained. For 100 mg tablets, in some embodiments, hardness is at least 4 kP, or in some embodiments, about 5 kP, or in some embodiments, at least about 6 kP. For 200 mg tablets, hardness is in some embodiments, at least 7 kP, or in some embodiments, at least about 9 kP, or in some embodiments about 11 kP. The mixture, however, is not to be compressed to such a degree that there is subsequent difficulty in achieving hydration when exposed to gastric fluid.

Tablet friability is, in some embodiments, less than about 1.0%, or in some embodiments, less than 0.8%, or in some embodiments, less than about 0.5% in a standard test.

Wet granulation, dry granulation or direct compression or encapsulation methods can be employed to prepare tablet or capsule compositions of the invention.

In some embodiments, the composition comprises milled or micronized agents. In some embodiments, conventional mills or grinders used impact milling such as pin milling of the agent provides improved blend uniformity to the final composition relative to other types of milling. Cooling of the material being milled, for example, using liquid nitrogen may be accomplished during milling to avoid heating the agent to undesirable temperatures.

The milled or micronized agent, if any, in some embodiments, may be blended with a desired amount of other components of the composition. In some embodiments, the agent is blended with one or more diluent(s), disintegrant(s) and/or binding agent(s) and, optionally, one or more wetting agent(s) in this step. In some embodiments, for tablet formulations it may be desirable to add microcrystalline cellulose following drying to improve compressibility of the granules and hardness of tablets prepared from the granules.

In some embodiments, water, e.g. purified water, is added to the dry powder mixture and the mixture is blended for an additional period of time, to form a wet granulated mixture. In some embodiments, a wetting agent is used, which in some embodiments is first added to the water and mixed for at least 15 minutes, or in some embodiments, at least 20 minutes, prior to adding the water to the dry powder mixture. In some embodiments, water is added to the mixture at once, gradually over a period of time, or in several portions over a period of time. In some embodiments, water is added gradually over a period of time. In some embodiments, the wetting agent is added to the dry powder mixture and water can is added to the resulting mixture. In some embodiments, an additional period of mixing after water addition is complete is accomplished to ensure uniform distribution of water in the mixture.

In some embodiments, wet granulated mixture is then wet milled, for example with a screening mill, to eliminate large agglomerates of material that form as a by-product of the wet granulation operation.

In some embodiments, wet granulated or wet milled mixture is then dried, for example, in an oven or a fluid bed dryer, for example a fluid bed drier, to form dry granules. In some embodiments, wet granulated mixture is extruded or spheronized prior to drying.

In some embodiments, dry granules are then reduced in size in preparation for compression or encapsulation. Conventional particle size reduction equipment such as oscillators or impact mills (such as Fitz mills) can be employed.

A slight decrease in granule size has been observed as mixing time increases for mixtures containing lower water amounts. It is hypothesized that where water concentration is too low to fully activate the binding agent employed, cohesive forces between the primary particles within the granules are insufficient to survive the shearing forces generated by the mixing blades and granule size attrition rather than growth occurs. Conversely, increasing the amount of water to fully activate the binding agent allows cohesive forces between the primary particles to survive the shearing forces generated by the mixing blades and granule growth rather than attrition occurs with increased mixing time and/or water addition rate. Variation of the screen size of the wet mill tends to have a greater impact on the granule size than variation of the feed rate and/or mill speed.

In some embodiments, dry granules are then placed in a suitable blender, such as a twin-shell blender, and optionally a lubricant (such as magnesium stearate) and any additional carrier materials are added (such as extragranular microcrystalline cellulose and/or extragranular croscarmellose sodium in certain tablet formulations) to form a final blended mixture. In some embodiments, where the diluents include microcrystalline cellulose, the addition of a portion of the microcrystalline cellulose during this step has been found to materially increase granule compressibility and tablet hardness.

In some embodiments, the final blended mixture is encapsulated (or, if tablets are to be prepared, compressed into tablets of the desired weight and hardness using appropriately sized tooling). Conventional compression and encapsulation techniques known in the art can be employed. Suitable results are obtained for capsules by employing bed heights ranging, in some embodiments, from about 20 mm to about 60 mm, compaction settings ranging from about 0 to about 5 mm, and speeds from about 60,000 capsules per hour to about 130,000 capsules per hour. Where coated tablets are desired, conventional coating techniques known in the art can be employed.

In some embodiments, this combination of operations produces granules that are uniform in drug content at the unit dose level, that readily disintegrate, that flow with sufficient ease so that weight variation can be reliably controlled during capsule filling or tableting, and that are dense enough in bulk so that the batch can be processed in the selected equipment and individual doses fit into the specified capsules or tablet dies.

In some embodiments, any of the compositions of this invention will comprise the agents as described, in any form or embodiment as described herein. In some embodiments, any of the compositions of this invention will consist of the agents as described, in any form or embodiment as described herein. In some embodiments, of the compositions of this invention will consist essentially of the agents as described, in any form or embodiment as described herein. In some embodiments, the term “comprise” refers to the inclusion of the indicated active agent, as well as inclusion of other active agents, and pharmaceutically acceptable carriers, excipients, emollients, stabilizers, etc., as are known in the pharmaceutical industry. In some embodiments, the term “consisting essentially of” refers to a composition, whose only active ingredient is the indicated active ingredient, however, other compounds may be included which are for stabilizing, preserving, etc. the formulation, but are not involved directly in the therapeutic effect of the indicated active ingredient. In some embodiments, the term “consisting essentially of” may refer to components which facilitate the release of the active ingredient. In some embodiments, the term “consisting” refers to a composition, which contains the active ingredient and a pharmaceutically acceptable carrier or excipient.

In one embodiment, the present invention provides combined preparations. In one embodiment, the term “a combined preparation” defines especially a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. In one embodiment, the combined preparation can be varied, e.g., in order to cope with the needs of a patient subpopulation to be treated or the needs of the single patient which different needs can be due to a particular disease, severity of a disease, age, sex, or body weight as can be readily made by a person skilled in the art. A kit of parts can include materials for forming a combined composition of a CPAM agent and a coagulation factor/hemostatic factor and/or a second anti-inflammatory agent, or can include a combination of such agents in separate unit dosage containers (or means for preparing separate unit dosages thereof—e.g., by resuspension or formulation of a form of the drug, such as a lyophilized form) for separate administration.

An agent (e.g., a CPAM agent) described herein typically will be understood as being at least substantially isolated and/or purified (by any suitable convention means) or isolated within commonly acceptable levels of purity for a comparable pharmaceutical agent.

It is to be understood that this invention is directed to, i.a., compositions and combined therapies as described herein, for any disease, disorder or condition, as appropriate, as will be appreciated by one skilled in the art. Certain applications of such compositions and combined therapies have been described hereinabove, for specific diseases, disorders and conditions, representing embodiments of this invention, and methods of treating such diseases, disorders and conditions in a subject by administering the agent, which abrogates or diminishes activation of a complement pathway as herein described, alone or as part of the combined therapy or using the compositions of this invention represent additional embodiments of this invention.

As noted elsewhere, in one aspect the invention relates to methods for treating synovitis, such as the treatment of synovitis in the context of a bleeding condition and/or in a patient suffering from a bleeding disorder, for example hemophilia. In one embodiment, the synovitis is acute and/or pre-acute, or in another embodiment, the synovitis is chronic.

In some embodiments, the synovitis, and/or therapeutic effect can be monitored by any means known in the art, including, but not limited to, ultrasonography and/or magnetic resonance imaging of the affected joint, as well as assessment of the Ritchie index, swollen joint count and score, swollen joint count of hands and wrists, evaluation of morning stiffness, pain intensity, Disease Activity Score (DAS), erythrocyte sedimentation rate, and C-reactive protein. In some embodiments, the therapeutic regimen may be modulated as a function of responsiveness to clinical intervention, for example, dose, administration route and/or timing may be varied, as well as the combination therapy altered or adjusted to optimize therapy in a subject.

In one embodiment, this invention provides a method of treating, reducing the incidence of, reducing the severity of or delaying onset (and/or delaying advancement of) of bleeding-associated inflammation in a subject, the method comprising administering to the subject an agent, which abrogates or diminishes activation of a complement pathway in said subject.

In some embodiments, this invention provides compositions for treating, and methods of treatment of, a variety of conditions associated (directly or indirectly in a subject) with bleeding (or significant and/or imminent risk thereof) or blood and inflammation, including systemic lupus erythematosus, rheumatoid arthritis, glomerulonephritis, myositis, neuralgia, synovitis, gout, ankylosing spondylitis, bursitis, or others, as will be appreciated by one skilled in the art. In one embodiment, such a disorder is treated in a patient suffering from a specific bleeding condition (e.g., from a wound, surgery, trauma, etc.) and/or a bleeding-related disease, disorder, or condition, such as hemophilia in addition to an inflammation disorder.

In some embodiments, this invention provides compositions for treating, and methods of treatment of, an inflammation-related disorder associated with blood, bleeding, or risk thereof, such as abnormal wound healing, acute injury to the eye tissue, angiogenesis-related disorders, angioplasty inflammation, ankylosing spondylitis, bacterial-induced inflammation, cerebral ischemia, Chlamydia-induced inflammation, coagulation, coronary artery bypass surgery inflammation, coronary artery disease, coronary plaque inflammation, Crohn's disease, endarterectomy inflammation, gastrointestinal bleeding, gingivitis, gout, gouty arthritis, head trauma, hemophilia, hereditary angioedema, hypoprothrombinernia, IBD related arthritis, idiopathic polymyositis, inflammation-related cardiovascular disorders, inflammatory bowel disease, irritable bowel syndrome, loosening of artificial joint implants, myocardial ischemia, myositis, nephritis, osteoarthritis, palindromic rheumatism, peptic ulcers, postoperative inflammation, pulmonary inflammation, recurrent gastrointestinal lesion, regional enteritis, retinitis, retinopathies, revascularization procedure inflammation, rheumatoid arthritis, sarcoidosis, scleritis, sclerodoma, stent placement inflammation, stroke ischemia, swelling occurring after injury, synovitis, systemic lupus erythematosus, systemic rheumatoid vasculitis, systemic sclerosis, tendonitis, thyroiditis, tissue ulceration, ulcerative colitis, vasculitis, or others. The invention also provides a method for promoting the sale of a CPAM agent, alone or in combination with a hemostatic factor/coagulation factor and/or a second anti-inflammatory agent, for the treatment of such diseases (e.g., by direct-to-consumer advertising, promotion via scientific liaisons, targeted advertising to key opinion leaders, etc.).

The compositions of this invention comprise and/or methods of this invention make use of agents, as described herein, to suppress, inhibit, treat, etc., bleeding-associated inflammation, which is a characteristic symptom and/or etiology of a number of diseases, including the listing hereinabove. In some embodiments, the compositions of this invention comprise and/or methods of this invention make use of agents to suppress angiogenesis, which in turn may compound, exacerbate or participate in disease severity. In some embodiments, such agents may comprise Anti-Invasive Factor, retinoic acid and derivatives thereof, paclitaxel, Suramin, Tissue Inhibitor of Metalloproteinase-1, Tissue Inhibitor of Metalloproteinase-2, Plasminogen Activator Inhibitor-1, Plasminogen Activator Inhibitor-2, and various forms of the lighter “d group” transition metals. These and other anti-angiogenic factors will be discussed in more detail below.

Anti-Invasive Factor, or “AIF” which is prepared from extracts of cartilage, contains constituents which are responsible for inhibiting the growth of new blood vessels. These constituents comprise a family of 7 low molecular weight proteins (<50,000 daltons) (Kuettner and Pauli, “Inhibition of neovascularization by a cartilage factor” in Development of the Vascular System, Pitman Books (CIBA Foundation Symposium 100), pp. 163-173, 1983), including a variety of proteins which have inhibitory effects against a variety of proteases (Eisentein et al, Am. J. Pathol. 81:337-346, 1975; Langer et al., Science 193:70-72, 1976; and Horton et al., Science 199:1342-1345, 1978). AIF suitable for use within the present invention may be readily prepared utilizing techniques known in the art (e.g., Eisentein et al, supra; Kuettner and Pauli, supra; and Langer et al., supra). Purified constituents of AIF such as Cartilage-Derived Inhibitor (“CDI”) (see Moses et al., Science 248:1408-1410, 1990) may also be readily prepared and utilized within the context of the present invention.

Retinoic acids alter the metabolism of extracellular matrix components, resulting in the inhibition of angiogenesis. Addition of proline analogs, angiostatic steroids, or heparin may be utilized in order to synergistically increase the anti-angiogenic effect of transretinoic acid. Retinoic acid, as well as derivatives thereof which may also be utilized in the context of the present invention, may be readily obtained from commercial sources, including for example, Sigma Chemical Co. (# R2625).

Paclitaxel is a highly derivatized diterpenoid (Wani et al., J. Am. Chem. Soc. 93:2325, 1971) which has been obtained from the harvested and dried bark of Taxus brevifolia (Pacific Yew.) and Taxomyces Andreanae and Endophytic Fungus of the Pacific Yew (Stierle et al., Science 60:214-216, 1993). Generally, paclitaxel acts to stabilize microtubular structures by binding tubulin to form abnormal mitotic spindles. “Paclitaxel” (which should be understood herein to include analogues and derivatives such as, for example, TAXOL®, TAXOTERE®, 10-desacetyl analogues of paclitaxel and 3′N-desbenzoyl-3′N-t-butoxy carbonyl analogues of paclitaxel) may be readily prepared utilizing techniques known to those skilled in the art (see also WO 94/07882, WO 94/07881, WO 94/07880, WO 94/07876, WO 93/23555, WO 93/10076, U.S. Pat. Nos. 5,294,637, 5,283,253, 5,279,949, 5,274,137, 5,202,448, 5,200,534, 5,229,529, and EP 590267), or obtained from a variety of commercial sources, including for example, Sigma Chemical Co., St. Louis, Mo. (T7402—from Taxus brevifolia).

Suramin is a polysulfonated naphthylurea compound that is typically used as a trypanocidal agent. Briefly, Suramin blocks the specific cell surface binding of various growth factors such as platelet derived growth factor (“PDGF”), epidermal growth factor (“EGF”), transforming growth factor (“TGF-β”), insulin-like growth factor (“IGF-1”), and fibroblast growth factor (“βFGF”). Suramin may be prepared in accordance with known techniques, or readily obtained from a variety of commercial sources, including for example Mobay Chemical Co., New York. (see Gagliardi et al., Cancer Res. 52:5073-5075, 1992; and Coffey, Jr., et al., J. of Cell. Phys. 132:143-148, 1987).

Tissue Inhibitor of Metalloproteinases-1 (“TIMP”) is secreted by endothelial cells which also secrete MTPases. TIMP is glycosylated and has a molecular weight of 28.5 kDa. TIMP-1 regulates angiogenesis by binding to activated metalloproteinases, thereby suppressing the invasion of blood vessels into the extracellular matrix. Tissue Inhibitor of Metalloproteinases-2 (“TIMP-2”) may also be utilized to inhibit angiogenesis. Briefly, TIMP-2 is a 21 kDa nonglycosylated protein which binds to metalloproteinases in both the active and latent, proenzyme forms. Both TIMP-1 and TIMP-2 may be obtained from commercial sources such as Synergen, Boulder, Colo.

Plasminogen Activator Inhibitor-1 (PA) is a 50 kDa glycoprotein which is present in blood platelets, and can also be synthesized by endothelial cells and muscle cells. PAI-1 inhibits t-PA and urokinase plasminogen activator at the basolateral site of the endothelium, and additionally regulates the fibrinolysis process. Plasminogen Activator Inhibitor-2 (PAI-2) is generally found only in the blood under certain circumstances such as in pregnancy, and in the presence of tumors. Briefly, PAI-2 is a 56 kDa protein which is secreted by monocytes and macrophages. It is believed to regulate fibrinolytic activity, and in particular inhibits urokinase plasminogen activator and tissue plasminogen activator, thereby preventing fibrinolysis.

A wide variety of other anti-angiogenic factors may also be utilized within the context of the methods and/or compositions of the present invention. Representative examples include Platelet Factor 4 (Sigma Chemical Co., #F1385); Protamine Sulphate (Clupeine) (Sigma Chemical Co., #P4505); Sulphated Chitin Derivatives (prepared from queen crab shells), (Sigma Chemical Co., #C3641; Murata et al., Cancer Res. 51:22-26, 1991); Sulphated Polysaccharide Peptidoglycan Complex (SP-PG) (the function of this compound may be enhanced by the presence of steroids such as estrogen, and tamoxifen citrate); Staurosporine (Sigma Chemical Co., #S4400); Modulators of Matrix Metabolism, including for example, proline analogs {[(L-azetidine-2-carboxylic acid (LACA) (Sigma Chemical Co., #A0760)), cishydroxyproline, d,L-3,4-dehydroproline (Sigma Chemical Co., #D0265), Thiaproline (Sigma Chemical Co., #T063.1)], α,α-dipyridyl (Sigma Chemical Co., #D7505), β-aminopropionitrile fumarate (Sigma Chemical Co., #A3134)]}; MDL 27032 (4-propyl-5-(4-pyridinyl)-2(3H)-oxazolone; Merion Merrel Dow Research Institute); Methotrexate (Sigma Chemical Co., #A6770; Hirata et al., Arthritis and Rheumatism 32:1065-1073, 1989); Mitoxantrone (Polyerini and Novak, Biochem. Biophys. Res. Comm. 140:901-907); Heparin (Folkman, Bio. Phar. 34:905-909, 1985; Sigma Chemical Co., #P8754); Interferons (e.g., Sigma Chemical Co., #13265); 2 Macroglobulin-serum (Sigma Chemical Co., #M7151); ChIMP-3 (Pavloff et al., J. Bio. Chem. 267:17321-17326, 1992); Chymostatin (Sigma Chemical Co., #C7268; Tomkinson et al., Biochem J. 286:475-480, 1992); β-Cyclodextrin Tetradecasulfate (Sigma Chemical Co., #C4767); Eponemycin; Camptothecin; Fumagillin (Sigma Chemical Co., #F6771; Canadian Patent No. 2,024,306; Ingber et al., Nature 348:555-557, 1990); Gold Sodium Thiomalate (“GST”; Sigma:G4022; Matsubara and Ziff, J. Clin. Invest. 79:1440-1446, 1987); (D-Penicillamine (“CDPT”; Sigma Chemical Co., #P4875 or P5000(HCl)); β-1-anticollagenase-serum; α2-antiplasmin (Sigma Chem. Co.: A0914; Holmes et al., J. Biol. Chem. 262(4):1659-1664, 1987); Bisantrene (National Cancer Institute); Lobenzarit disodium (N-(2)-carboxyphenyl-4-chloroanthronilic acid disodium or “CCA”; Takeuchi et al., Agents Actions 36:312-316, 1992); Thalidomide; Angostatic steroid; AGM-1470; carboxynaminolmidazole; metalloproteinase inhibitors such as BB94 and the peptide CDPGYIGSR-NH2 (SEQUENCE ID NO. 1) (Iwaki Glass, Tokyo, Japan).

### EXAMPLES

The following examples are presented in order to more fully illustrate some embodiments of the invention. They should, in no way be construed, however, as limiting the scope of the invention.

### EXAMPLE 1

### C5aR Expression in Diseased Joints

Analysis of human joint expression of C5aR in normal and diseased states will be undertaken. Joint tissue is collected from adults undergoing synovectomy or total joint replacement, with tissue collected at different phases of synovitis, including acute, sub-acute and chronic synovitis. Written informed consent is obtained from the subjects.

### Collection of Joint Samples

Synovial tissue (1˜2 cm in diameter, several samples) is obtained from each knee undergoing surgery. Tissue sections are fixed in 4% phosphate buffed formalin for immunohistochemical studies. After fixation, tissues are processed, embedded in paraffin, sectioned and mounted to slides. Sections are also snap-frozen in liquid nitrogen and stored for immunohistochemistry analysis. Standard histologic processing and staining is performed, including H & E staining.

### Immunohistochemical Analysis:

Slides are de-paraffinized, rehydrated and endogenous peroxidase is quenched by standard protocols. Goat serum is used as a blocking agent and slides are probed with a mouse monoclonal antibody raised against human C5aR (W17/1). Murine IgG1 will be used as a control antibody. A biotinylated rat anti-mouse immunoglobulin is the secondary antibody used and slides are incubated in a humidified chamber. Horse Radish Peroxidase (HRP) is added as is an AEC substrate, and the reaction terminated by the addition of water.

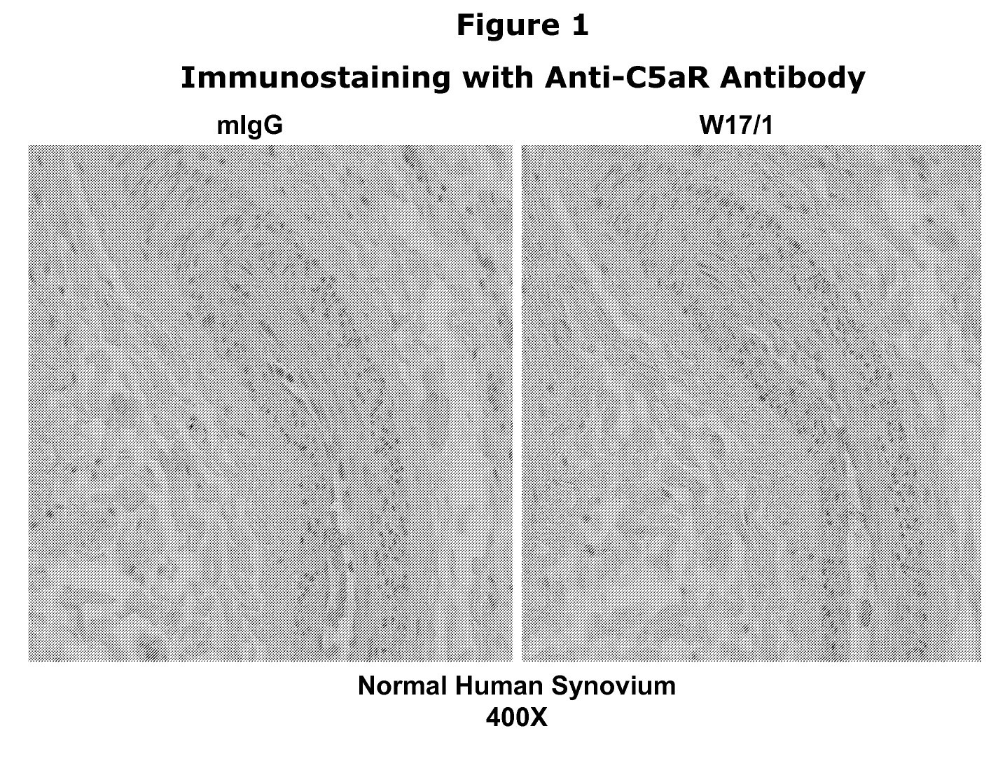

Slides are counter-stained with Mayer's hematoxylin, viewed and recorded. Normal joint tissue is purchased, and processed and probed similarly. An example of normal tissue staining is shown in FIG. 1 (which is further discussed in Example 4).

### RNA Analysis:

Frozen tissue samples are homogenized in Trizol Reagent followed by chloroform/Trizol extraction. Aqueous phases are subjected to 1:2 isopropanol/Trizol extractions for RNA precipitation. RNA pellets are washed with an ethanol/Trizol cocktail, air dried, and re-dissolved in DEPC water.

RNA may also be extracted from formalin fixed tissues by standard methodology.

C5aR expression will thus be determined in diseased versus healthy tissue quantitatively by RT-PCR.

It is expected that C5aR expression will be detected in synovial tissues and will be up-regulated in the synovial tissues taken from the synovitis patients compared to normal control, indicating that the receptor is modulated in the disease pathogenesis.

### EXAMPLE 2

### Elimination of C5aR and Development of Synovitis

C5ar1^{tm1Cge} mice genetically disrupted for C5aR expression will be crossed with F8t^{m1Kaz} mice genetically disrupted for Factor VIII expression, to yield mice deficient in expression of either protein. Confirmation of the genotype will be accomplished via Southern blotting or RT-PCR. Induction of hemophilic synovitis will be performed in these mice, as described in Hakobyan N. et al., Haemophilia. 2005 May; 11 (3): 227-32 and/or Valentino L A, et al., Haemophilia. 2004 May; 10 (3):280-7. The effect of the absence of C5aR on the development of synovitis will be assessed, and it will be evaluated whether the double knock out mice are more susceptible to joint bleeding induction compared to Factor VIII knock out mice. It is expected that the double knock out mice will be less susceptible to joint bleeding due to the absence of C5a/C5aR interaction.

### EXAMPLE 3

### Development of Agents Which Suppress C5aR

Monoclonal Antibodies, which specifically bind and block C5aR will be developed by standard methodology. In some cases, C57BL/6 mice are immunized intra-peritoneally (IP) every 14 days for 6 times followed by a single intravenous (IV) injection 4-5 days after the final IP injection with L1.2/human C5aR expressing cells activated for 24 hours with butyric acid and treated with mitomycin C. Mice are then sacrificed and their spleen cells are fused with Sp2/0 myeloma cells, with resulting hybridomas screened by FACS, and hybridomas producing monoclonal antibodies to C5aR are identified and isolated.

In other embodiments, monoclonal antibodies specifically interacting with human C5aR may be produced according to methods outlined in Lee H. et al., 2006 Nature Biotechnology 24: 1279-1284, or the antibodies produced therein may be utilized.

Once candidate monoclonal antibodies have been produced, their ability to block ligand binding and/or neutrophil chemotaxis is evaluated as well. Lead antibodies are identified and tested in mouse models of synovitis, for example as described in Example 2.

Human clinical trials may be undertaken as well, in adult subjects with hemophilic synovitis. Monoclonal antibodies inhibiting C5aR mediated signaling may be administered to the subject, including so-called “humanized” versions of the antibodies described herein. Therapeutic effects of the antibody are documented. It is expected that such agents will provide useful physiological, prophylactic, and/or therapeutic effects as described herein.

### EXAMPLE 4

### Immunohistological Staining with Synovial Tissue and anti-C5aR Antibody

Normal synovium was purchased from Asterand plc (Detroit, Mich., USA; Royston, Herts, UK). It was excised from an 82 year old diseased male Caucasian. The synovial tissues were processed into formalin-fixed, paraffin-embedded sections (5-10 micron thick) by Asterand.

For staining, the mouse anti-human C5aR1 W17/1 monoclonal antibody (Abcam—Cambridge, Mass., USA) was used as primary antibody at a concentration of 10 μg/ml. The biotinylated rat anti-mouse IgG1 (BD Pharmingen) was used as secondary antibody. Streptavidin conjugated HRP (BD Pharmingen) and AEC substrate (BD Pharmingen) were used for color development. The slides was counter stained with Mayer's hematoxylin (Sigma) and mounted for viewing. The results are shown in FIG. 1. As shown in FIG. 1, normal synovial tissue appears to express a minimal level of C5aR.

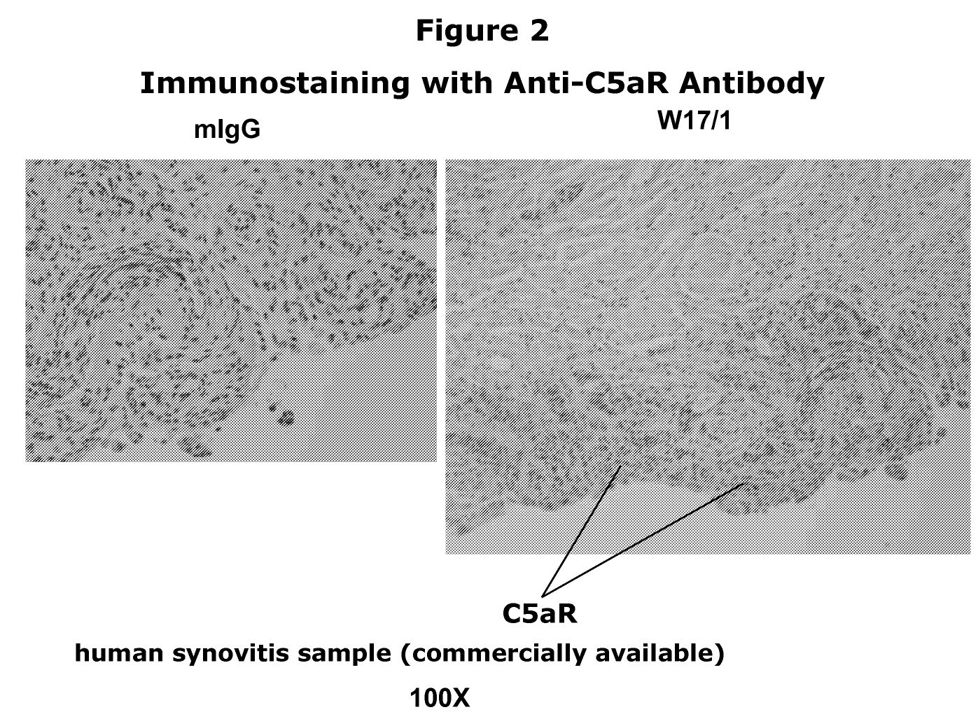

Additional synovial tissues, taken from a 65 year old diseased female with ongoing osteoarthritis of right knee at the time of excision, were purchased from Asterand, Inc. (Detroit, Mich., USA). The same antibody (W17/1) was used under the same conditions as described above with respect to FIG. 1 to stain the tissue. The results are shown in FIG. 2 (as FIG. 2 is a black-and-white reproduction of the original color micrograph it is difficult to see areas of differential staining; indicator lines have been added to the figure to point to the area where C5aR was detected). The results of this experiment reflect that in comparison to normal synovium, the human osteoarthritis synovium expresses moderate level of C5aR.

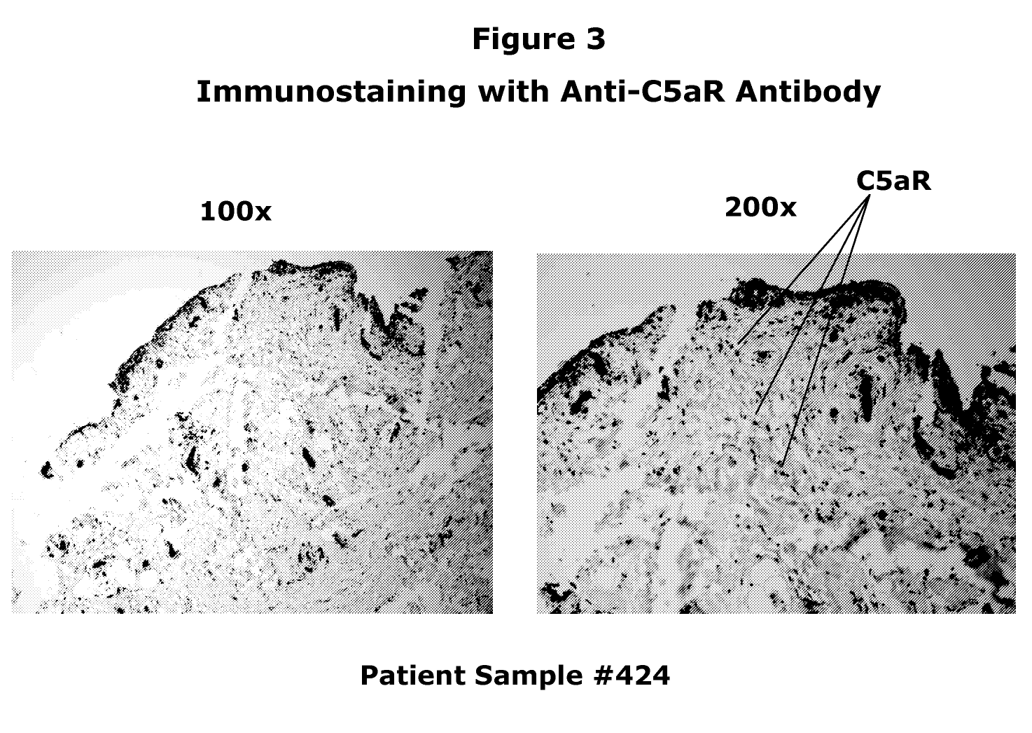

A further study was performed as follows. A patient joint sample was obtained from Rush University (Chicago, Ill., USA). The sample was described as a joint sample from a hemophilic synovitis patient undergoing synovectomy. Immunohistochemistry was performed on paraffin-embedded human synovial tissues. Serial 5-μm sections were mounted on glass slides. Slides were de-waxed by placement in a 56-60° C. oven for 1 h and then transferred to a xylene bath. Slides were then rehydrated and heat-induced epitope retrieval (HIER) was then performed. Slides were rinsed with PBS and then endogenous peroxidase was blocked with 3% hydrogen peroxide. Slides were incubated overnight at 4° C. with primary antibodies, washed three times with PBS/0.1% Tween and then incubated with anti-mouse-HRP polymer (DAKO, undiluted) for 30 min at room temperature. After washing in PBS, slides were rinsed in deionized water and visualized with a permanent AEC chromogen (BioCare Medical) according to manufacturer's directions. Slides were counterstained with Mayer's Hematoxylin (Sigma), dehydrated through alcohols, cleared in xylene, and coverslipped with Permount (Fisher). Primary antibodies tested were mouse anti-human C5aR, W17/1 (Abcam, 0.1 mg/mL) at 1:100 dilution. Undiluted mouse IgG was used as negative control (Dako, 6.1 mg/mL). The results of this work are presented in FIG. 3. As can be seen in FIG. 3, clear staining with anti-C5aR was shown in the diseased tissue.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context.

Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims and/or aspects included herein as permitted by applicable law.

1. A method for treating synovitis in a subject having synovitis comprising administering to the subject an agent that inhibits or diminishes activation of a C5a receptor (C5aR).

2. The method according to claim 1, wherein the agent is an antibody specific for C5aR.

3. The method according to claim 1, wherein the subject has a condition that causes hemophilia.

4. The method according to claim 2, wherein the agent specifically binds an extracellular loop of a C5aR.

5. The method according to claim 2, wherein the antibody is expressed by or comprises the functional portion of an antibody expressed by a hybridoma having an ECACC accession number 00110609, 02090226, or 02090227, or ATCC accession number HB 11382, HB 11384, or HB-11625.

6. The method according to claim 1, wherein the synovitis is acute or sub-acute, or chronic.

7. The method of claim 1, wherein the method comprises administering a coagulation factor or hemostatic factor.

8. The method according to claim 7, wherein the coagulation factor or hemostatic factor is selected from the group consisting of Factor XIII, Factor IX, Factor X, Factor XI, Factor VII, Factor VIII, fibrinogen, thrombin, a variant of any thereof, a derivative of any thereof, and combinations of any thereof.

9. A method for reducing bleeding-associated inflammation in a subject comprising administering to the subject an agent that diminishes activation of C5aR.

10. The method according to claim 9, wherein the agent is an antibody specific for C5aR.

11. The method according to claim 10, wherein the agent specifically binds an extracellular loop of a C5aR.

12. The method according to claim 10, wherein the antibody is expressed by or comprises the functional portion of an antibody expressed by a hybridoma having an ECACC accession number 00110609, 02090226, or 02090227, or ATCC accession number HB 11382, HB 11384, or HB-11625.

13. The method of claim 1, wherein the subject is a human patient.

14. The method of claim 2, wherein the subject is a human patient.

15. The method of claim 4, wherein the subject is a human patient.

16. The method of claim 5, wherein the subject is a human patient.

17. The method of claim 16, wherein the synovitis is acute or sub-acute, or chronic.

18. The method of claim 9, wherein the subject is a human patient.

19. The method of claim 11, wherein the subject is a human patient.

20. The method of claim 12, wherein the subject is a human patient.
